(19) United States
(12) Patent Application Publication
(10) Pub. No.: US 2020/0181134 A1
(43) Pub. Date: Jun. 11, 2020

(54) Antiparasitic Compounds

(71) Applicant: Avista Phama Solutions, Inc., Durham, NC (US)

(72) Inventor: Jason D. Speake, Winston-Salem, NC (US)

(21) Appl. No.: 16/315,778

(22) PCT Filed: Jul. 7, 2017
(86) PCT No.: PCT/US17/41029

§ 371 Date: Jan. 7, 2019

Related U.S. Application Data
(60) Provisional application No. 62/359,719, filed on Jul. 8, 2016.

(51) Int. Cl. C07D 417/14; A61P 33/14

(52) U.S. Cl. CPC C07D 417/14; A61P 33/14

(57) ABSTRACT

The present invention relates to isothiazoline compounds of formula (I). The compounds are useful for combating or controlling invertebrate pests, in particular arthropod pests and nematodes. The invention also relates to a method for controlling invertebrate pests by using these compounds and to veterinary compositions comprising said compounds.

### CROSS REFERENCE TO RELATED APPLICATIONS

This application claims the benefit of U.S. Provisional Patent Application No. 62/359,719, filed Jul. 8, 2016, the entire contents of which are hereby incorporated herein by reference.

### FIELD OF THE INVENTION

The present invention includes spirocyclic derivatives having parasiticidal activity. The present invention preferably includes spirocyclic azetidenyl-isobenzofuran derivatives having an isothiazoline moiety. The present invention also includes processes of making said spirocyclic derivatives, compositions comprising said spirocyclic derivatives, and methods of use thereof.

### BACKGROUND

There is a need for improved antiparasitics, and in particular there is a need for improved insecticides and acaricides, particularly for use in animal health. Furthermore, there is a need for improved topical and oral products with convenient administration. Still further, there is a need for improved compositions which contains one or more active antiparasitics, which can be used to effectively treat against parasites. Such improvements would be particularly useful for the treatment of animals including: birds (e.g., chickens and turkeys), fish, companion animals (e.g., cats, dogs, llamas, and horses), and livestock (e.g., cattle, bison, swine, sheep, deer, elk, and goats).

Currently available insecticidal and acaricidal treatments for animals do not always demonstrate good activity, good speed of action, or a long duration of action. Most treatments contain hazardous chemicals that can have serious consequences, including neurotoxicity and lethality from accidental ingestion. Persons applying these agents are generally advised to limit their exposure. Pet collars and tags have been utilized to overcome some problems, but these are susceptible to chewing, ingestion, and subsequent toxicological effects to the animal. Thus, current treatments achieve varying degrees of success, which depend partly on toxicity, method of administration, and efficacy. Additionally, some currently available agents are becoming ineffective due to parasitic resistance.

Despite the availability of effective, broad spectrum antiparasitics, there remains a need for safer and more convenient, efficacious, and environmentally friendly products that will overcome the ever-present threat of resistance development. The present invention includes new isothiazoline spiroazetidinyl-isobenzofuran derivatives which demonstrate such properties.

### SUMMARY

The present invention includes compounds according to Formula (I), including stereoisomers, and pesticidal, veterinary, or pharmaceutically acceptable salts thereof:

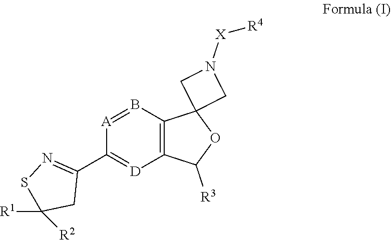

wherein:
R¹ is optionally substituted aryl or optionally substituted heteroaryl;
R² is alkyl or haloalkyl;
each of A, B, and D individually is CR⁵ or N;
each R⁵ individually is hydrogen, alkyl, halogen, haloalkyl, or aryl;
X is L¹-L²-L³;
each of L¹, L², and L³ individually is bond, C(O), (CH₂)ₙ, SO₂, O, or NH;
n is 1 to 6;
R³ is hydrogen, alkyl, or ═O; and
R⁴ is hydrogen, optionally substituted alkyl, optionally substituted haloalkyl, optionally substituted cycloalkyl, optionally substituted heterocyclyl, optionally substituted aryl, or optionally substituted heteroaryl;
or a pesticidally, veterinarily, or pharmaceutically acceptable salt thereof.

One embodiment of the invention includes compositions comprising a compound of formula (I) along with a pesticidally acceptable carrier. The compositions of the invention can also be in a variety of forms which include, but are not limited to, oral formulations, injectable formulations, and topical, dermal, or subdermal formulations. The formulations are intended to be administered to an animal, which includes, but is not limited to, mammals, birds, and fish. Examples of mammals include, but are not limited to, humans, cattle, sheep, goats, llamas, alpacas, pigs, horses, donkeys, dogs, cats, and other livestock or domestic mammals. Examples of birds include turkeys, chickens, ostriches, and other livestock or domestic birds.

The present invention includes compositions comprising a compound of formula (I) suitable for treatment of a locus that may be infected with parasites, such as a plant or animal such as a mammal, or for the prevention of infection or infestation of a locus with parasites.

Another embodiment of the present invention includes combination therapy, whereby one or more compounds of formula (I) can be employed as such or in the form of their preparations or formulations as combinations with one or more other pesticidally active substances, such as, for example, insecticides, attractants, sterilants, nematicides, acaricides, fungicides, herbicides, and with safeners, fertilizers, or growth regulators. The combinations may be part of the same formulation, or may be administered separately or sequentially to the locus.

Another embodiment of the present invention includes a compound of formula (I), or a composition comprising a compound of formula (I), for use in treating or preventing parasitic infection or infestation.

Another embodiment of the present invention includes the use of a compound of formula (I) for the manufacture of a medicament for use in treating or preventing parasitic infection or infestation.

Another embodiment of the present invention includes a method of treating or preventing a parasitic infection comprising the administration of an effective amount of a compound of formula (I), or a composition comprising a compound of formula (I) to a locus.

One embodiment of the present invention is a compound of the present invention selected from:

1-[6-[5-(6-chloro-3-pyridyl)-5-(trifluoromethyl)-4H-isothiazol-3-yl]spiro[1H-isobenzofuran-3,3′-azetidine]-t-yl]-3,3,3-trifluoro-propan-1- one;
1-[6-[5-(6-chloro-3-pyridyl)-5-(trifluoromethyl)-4H-isothiazol-3-yl]spiro[1H-isobenzofuran-3,3′-azetidine]-1′-yl]-2-methylsulfonyl- ethanone;
1-[6-[5-(6-chloro-3-pyridyl)-5-(trifluoromethyl)-4H-isothiazol-3-yl]spiro[1H-isobenzofuran-3,3′-azetidine]-1′-yl]-2-methyl-propan-1-one;
6-[5-(6-chloro-3-pyridyl)-5-(trifluoromethyl)-4H-isothiazol-3-yl]-N-ethyl-spiro[1H-isobenzofuran-3,3′-azetidine]-1′-carboxamide;
6-[5-(6-chloro-3-pyridyl)-5-(trifluoromethyl)-4H-isothiazol-3-yl]-1′-ethylsulfonyl-spiro[1H-isobenzofuran-3,3′-azetidine];
1-[6-[5-(6-Chloro-2-pyridyl)-5-(trifluoromethyl)-4H-isothiazol-3-yl]spiro[1H-isobenzofuran-3,3′-azetidine]-1′-yl]-3,3,3- trifluoropropan-1-one;
1-[6-[5-(6-Chloro-2-pyridyl)-5-(trifluoromethyl)-4H-isothiazol-3-yl]spiro[1H-isobenzofuran-3,3′-azetidine]-1′-yl]-2- methylsulfonylethanone;
1-[6-[5-(6-Chloro-2-pyridyl)-5-(trifluoromethyl)-4H-isothiazol-3-yl]spiro[1H-isobenzofuran-3,3′-azetidine]-1′-yl]-2-methylpropan-1-one;
6-[5-(6-Chloro-2-pyridyl)-5-(trifluoromethyl)-4H-isothiazol-3-yl]-N-ethylspiro[1H-isobenzofuran-3,3′-azetidine]-1′-carboxamide;
6-[5-(6-Chloro-2-pyridyl)-5-(trifluoromethyl)-4H-isothiazol-3-yl]-1′-ethylsulfonylspiro[1H-isobenzofuran-3,3′-azetidine];
6-[5-(3,5-dichlorophenyl)-5-(trifluoromethyl)-4H-isothiazol-3-yl]-N-ethyl-spiro[1H-isobenzofuran-3,3′-azetidine]-1′-carboxamide;
1-[6-[5-(3,5-dichlorophenyl)-5-(trifluoromethyl)-4H-isothiazol-3-yl]spiro[1H-isobenzofuran-3,3′-azetidine]-1′-yl]-3,3,3-trifluoro-propan-1-one;
1-[6-[5-(3,5-dichlorophenyl)-5-(trifluoromethyl)-4H-isothiazol-3-yl]spiro[1H-isobenzofuran-3,3′-azetidine]-1′-yl]-2-methyl-propan-1- one;
1-[6-[5-(3,5-dichlorophenyl)-5-(trifluoromethyl)-4H-isothiazol-3-yl]spiro[1H-isobenzofuran-3,3′-azetidine]-1′-yl]-2-methylsulfonyl-ethanone;
6-[5-(3,5-dichlorophenyl)-5-(trifluoromethyl)-4H-isothiazol-3-yl]-1′-ethylsulfonyl-spiro[1H-isobenzofuran-3,3′-azetidine];
1-[6-[5-(3,5-dichloro-4-fluoro-phenyl)-5-(trifluoromethyl)-4H-isothiazol-3-yl]spiro[1H-furo[3,4-c]pyridine-3,3′-azetidine]-1′-yl]- 3,3,3-trifluoro-propan-1-one;
1-[6-[5-(3,5-dichloro-4-fluoro-phenyl)-5-(trifluoromethyl)-4H-isothiazol-3-yl]spiro[1H-furo[3,4-c]pyridine-3,3′-azetidine]-1′-yl]- 2-methyl-propan-1-one;
1-[6-[5-(3,5-dichloro-4-fluoro-phenyl)-5-(trifluoromethyl)-4H-isothiazol-3-yl]spiro[1H-furo[3,4-c]pyridine-3,3′-azetidine]-1′-yl]- 2-methylsulfonyl-ethanone;
6-[5-(3,5-dichloro-4-fluoro-phenyl)-5-(trifluoromethyl)-4H-isothiazol-3-yl]-1′-ethylsulfonyl-spiro[1H-furo[3,4-c]pyridine-3,3′-azetidine];
6-[5-(3,5-dichloro-4-fluoro-phenyl)-5-(trifluoromethyl)-4H-isothiazol-3-yl]-N-ethyl-spiro[1H-furo[3,4-c]pyridine-3,3′-azetidine]-1′- carboxamide;
1-[2-[5-(3,5-dichloro-4-fluoro-phenyl)-5-(trifluoromethyl)-4H-isothiazol-3-yl]spiro[7H-furo[3,4-b]pyridine-5,3′-azetidine]-1′-yl]- 3,3,3-trifluoro-propan-1-one;
1-[2-[5-(3,5-dichloro-4-fluoro-phenyl)-5-(trifluoromethyl)-4H-isothiazol-3-yl]spiro[7H-furo[3,4-b]pyridine-5,3′-azetidine]-1′-yl]- 2-methyl-propan-1-one;
1-[2-[5-(3,5-dichloro-4-fluoro-phenyl)-5-(trifluoromethyl)-4H-isothiazol-3-yl]spiro[7H-furo[3,4-b]pyridine-5,3′-azetidine]-1′-yl]- 2-methylsulfonyl-ethanone;
2-[5-(3,5-dichloro-4-fluoro-phenyl)-5-(trifluoromethyl)-4H-isothiazol-3-yl]-1′-ethylsulfonyl-spiro[7H-furo[3,4-b]pyridine-5,3′-azetidine];
2-[5-(3,5-dichloro-4-fluoro-phenyl)-5-(trifluoromethyl)-4H-isothiazol-3-yl]-N-ethyl-spiro[7H-furo[3,4-b]pyridine-5,3′-azetidine]-1′- carboxamide;
1-[3-[5-(3,5-dichloro-4-fluoro-phenyl)-5-(trifluoromethyl)-4H-isothiazol-3-yl]spiro[5H-furo[3,4-b]pyridine-7,3′-azetidine]-1′-yl]- 3,3,3-trifluoro-propan-1-one;
1-[3-[5-(3,5-dichloro-4-fluoro-phenyl)-5-(trifluoromethyl)-4H-isothiazol-3-yl]spiro[5H-furo[3,4-b]pyridine-7,3′-azetidine]-1′-yl]- 2-methyl-propan-1-one;
1-[3-[5-(3,5-dichloro-4-fluoro-phenyl)-5-(trifluoromethyl)-4H-isothiazol-3-yl]spiro[5H-furo[3,4-b]pyridine-7,3′-azetidine]-1′-yl]- 2-methylsulfonyl-ethanone;
3-[5-(3,5-dichloro-4-fluoro-phenyl)-5-(trifluoromethyl)-4H-isothiazol-3-yl]-1′-ethylsulfonyl-spiro[5H-furo[3,4-b]pyridine-7,3′-azetidine];
3-[5-(3,5-dichloro-4-fluoro-phenyl)-5-(trifluoromethyl)-4H-isothiazol-3-yl]-N-ethyl-spiro[5H-furo[3,4-b]pyridine-7,3′-azetidine]-1′- carboxamide;
1-[6-[5-(2,6-dichloro-4-pyridyl)-5-(trifluoromethyl)-4H-isothiazol-3-yl]spiro[1H-isobenzofuran-3,3′-azetidine]-1′-yl]-3,3,3-trifluoro- propan-1-one;
1-[6-[5-(2,6-dichloro-4-pyridyl)-5-(trifluoromethyl)-4H-isothiazol-3-yl]spiro[1H-isobenzofuran-3,3′-azetidine]-1′-yl]-2-methylsulfonyl- ethanone;
1-[6-[5-(2,6-dichloro-4-pyridyl)-5-(trifluoromethyl)-4H-isothiazol-3-yl]spiro[1H-isobenzofuran-3,3′-azetidine]-1′-yl]-2-methyl-propan- 1-one;
6-[5-(2,6-dichloro-4-pyridyl)-5-(trifluoromethyl)-4H-isothiazol-3-yl]-N-ethyl-spiro[1H-isobenzofuran-3,3′-azetidine]-1′-carboxamide;
6-[5-(2,6-dichloro-4-pyridyl)-5-(trifluoromethyl)-4H-isothiazol-3-yl]-1′-ethylsulfonyl-spiro[1H-isobenzofuran-3,3′-azetidine];
1-[6-[5-(3,5-dichlorophenyl)-5-methyl-4H-isothiazol-3-yl]spiro[1H-isobenzofuran-3,3′-azetidine]-1′-yl]-3,3,3-trifluoro-propan-1-one;
1-[6-[5-(3,5-dichlorophenyl)-5-methyl-4H-isothiazol-3-yl]spiro[1H-isobenzofuran-3,3′-azetidine]-1′-yl]-2-methyl-propan-1-one;
1-[6-[5-(3,5-dichlorophenyl)-5-methyl-4H-isothiazol-3-yl]spiro[1H-isobenzofuran-3,3′-azetidine]-1′-yl]-2-methylsulfonyl-ethanone;
6-[5-(3,5-dichlorophenyl)-5-methyl-4H-isothiazol-3-yl]-1′-ethylsulfonyl-spiro[1H-isobenzofuran-3,3′-azetidine];
6-[5-(3,5-dichlorophenyl)-5-methyl-4H-isothiazol-3-yl]-N-ethyl-spiro[1H-isobenzofuran-3,3′-azetidine]-1′-carboxamide;
1-[6-[5-(3,5-dichloro-4-fluoro-phenyl)-5-methyl-4H-isothiazol-3-yl]spiro[1H-isobenzofuran-3,3′-azetidine]-1′-yl]-3,3,3-trifluoro- propan-1-one;
1-[6-[5-(3,5-dichloro-4-fluoro-phenyl)-5-methyl-4H-isothiazol-3-yl]spiro[1H-isobenzofuran-3,3′-azetidine]-1′-yl]-2-methyl-propan-1-one;
1-[6-[5-(3,5-dichloro-4-fluoro-phenyl)-5-methyl-4H-isothiazol-3-yl]spiro[1H-isobenzofuran-3,3′-azetidine]-1′-yl]-2-methylsulfonyl- ethanone;
6-[5-(3,5-dichloro-4-fluoro-phenyl)-5-methyl-4H-isothiazol-3-yl]-1′-ethylsulfonyl-spiro[1H-isobenzofuran-3,3′-azetidine];
6-[5-(3,5-dichloro-4-fluoro-phenyl)-5-methyl-4H-isothiazol-3-yl]-N-ethyl-spiro[1H-isobenzofuran-3,3′-azetidine]-1′-carboxamide;
6′-[5-(3,5-dichloro-4-fluoro-phenyl)-5-(trifluoromethyl)-4H-isothiazol-3-yl]-1-(3,3,3-trifluoropropanoyhspiro[azetidine-3,3′-isobenzofuran]-1′-one;
6′-[5-(3,5-dichloro-4-fluoro-phenyl)-5-(trifluoromethyl)-4H-isothiazol-3-yl]-1-(2-methylpropanoyl)spiro[azetidine-3,3′-isobenzofuran]-1′-one;
6′-[5-(3,5-dichloro-4-fluoro-phenyl)-5-(trifluoromethyl)-4H-isothiazol-3-yl]-1-(2-methylsulfonylacetyl)spiro[azetidine-3,3′-isobenzofuran]-1′- one;
6′-[5-(3,5-dichloro-4-fluoro-phenyl)-5-(trifluoromethyl)-4H-isothiazol-3-yl]-1-ethylsulfonyl-spiro[azetidine-3,3′-isobenzofuran]-1′-one;
5′-[5-(3,5-dichloro-4-fluoro-phenyl)-5-(trifluoromethyl)-4H-isothiazol-3-yl]-N-ethyl-3′-oxo-spiro[azetidine-3,1′-isobenzofuran]-1-carboxamide;

or a pesticidally, veterinarily, or pharmaceutically acceptable salt thereof.

One embodiment of the present invention is a composition comprising a compound of the present invention and a pesticidally acceptable carrier. Another embodiment of the present invention is a combination comprising a compound of the present invention and one or more other pesticidally active substances. Another embodiment of the present invention is a method for controlling parasites at a locus comprising applying to the locus an effective amount of a compound of the present invention. Another embodiment of the present invention is a method of treating or preventing parasitic infection or infestation in a subject comprising administering to the subject an effective amount of a compound of the present invention. In one aspect, the parasite is a flea or tick. In one aspect, the parasite is Ctenocephalides felis, R. sanguineus, D. variablis, A. americanum, or I. scapularis. In one aspect, the parasite is a helminth. In one aspect, the parasite is Dirofilaria immitis. In one aspect the parasite is a mosquito. Another embodiment of the present invention is a compound of the present invention for use in treating or preventing parasitic infection or infestation. Another embodiment is a compound of the present invention for use in medicine.

### DETAILED DESCRIPTION

One or more aspects and embodiments may be incorporated in a different embodiment although not specifically described. That is, all aspects and embodiments can be combined in any way or combination.

### Definitions

When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, “alkyl” refers to monovalent saturated aliphatic hydrocarbyl groups having from 1 to 20 carbon atoms, preferably 1-8 carbon atoms, preferably 1-6 carbon atoms. The hydrocarbon chain can be either straight-chained or branched. Illustrative alkyl groups include methyl, ethyl, n-propyl, isopropyl, n-butyl, iso-butyl, and tert-butyl. Similarly, an “alkenyl” group refers to an alkyl group having one or more double bonds present in the chain.

As used herein, “cycloalkyl” refers to an unsaturated or partially saturated hydrocarbon ring, containing from 3 to 6 ring atoms. Illustrative cycloalkyl groups include cyclopropyl, cyclobutyl, cyclopentyl, cyclohexyl, as well as partially saturated versions thereof, such as cyclohexenyl, and cyclohexadienyl.

As used herein “halogen” or “halo” refers to a halogen. In some embodiments, the halogen is preferably Br, Cl, or F.

As used herein, “haloalkyl” refers to monovalent saturated aliphatic hydrocarbyl groups having from 1 to 20 carbon atoms, preferably 1-8 carbon atoms, preferably 1-6 carbon atoms, wherein at least one hydrogen atom is substituted by a halogen, including but not limited to perhalo groups where all hydrogen atoms are replaced with halogen atoms. The haloalkyl chain can be either straight-chained or branched. Illustrative alkyl groups include trifluoromethyl, trifluoroethyl, trifluoropropyl, trifluorobutyl, and pentafluoroethyl. Similarly, a “haloalkenyl” group refers to a haloalkyl group having one or more double bonds present in the chain.

As used herein “heterocyclyl” or “heterocycle” refers to an unsaturated or partially saturated ring containing from 3 to 6 ring atoms and from 1 to 4 heteroatoms, which may be the same or different, selected from nitrogen, oxygen and sulfur. Illustrative heterocyclyl groups include oxirane, tetrahydrofuranyl, morpholino, pyrrolidinyl, tetrahydrothiophene, dioxane, and piperidinyl.

As used herein “aryl” refers to an aromatic ring system containing from 5 to 10 ring atoms. Illustrative aryl groups include phenyl and naphthyl.

As used herein “heteroaryl” refers to an heteroaromatic ring system containing from 5 to 10 ring atoms and from 1 to 4 heteroatoms, which may be the same or different, selected from nitrogen, oxygen and sulfur. Illustrative heteroaryl groups include pyridyl (pyridinyl), furan, thiophene, pyrazolyl, tetrazolyl, oxazolyl, thiazolyl, imidazolyl, and pyrimidinyl.

As used herein “optionally substituted” refers to a substitution of a hydrogen atom, which would otherwise be present on the substituent. When discussing ring systems, the optional substitution is typically with 1, 2, or 3 substituents replacing the normally-present hydrogen. When referencing straight and branched moieties, however, the number of substitutions can be more, occurring wherever hydrogen is usually present. The substitutions can be the same or different. Illustrative substitutions include nitro, —NR′R″, cyano, —NR′COR′″, alkyl, alkenyl, —C(O), —SO₂R′″, —NR′SO₂R′″, —SO₂NR′R″, —CONR′R″, —CONHC₆H₅, hydroxy, alkoxy, alkylsulfonyl, haloalkyl, haloalkenyl, haloalkoxy, mercapto (—SH), thioalkyl, halogen, cycloalkyl, heterocyclyl, aryl, or heteroaryl, where R′ and R″ are the same or different and each represents hydrogen or alkyl; or when R′ and R″ are each attached to a nitrogen atom, they may form a saturated or unsaturated heterocyclic ring containing from 4 to 6 ring atoms, and wherein R′″ is alkyl or haloalkyl.

As used herein the phrase pesticidal or pesticidally, veterinary or veterinarily, or pharmaceutical or pharmaceutically acceptable salt refers to any salt of a compound disclosed herein which retains its biological properties and which is not toxic or otherwise undesirable for pesticidal, veterinary, or pharmaceutical use. Such salts may be derived from a variety of organic and inorganic counter-ions known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only, salts of non-toxic organic or inorganic acids, such as halides, such as , chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

In certain cases, the depicted substituents can contribute to optical and/or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. When a compound has an asymmetric center, for example when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed. Engl. 5:511); Prelog and Helmchen, 1982, Angew. Chem. 94: 614-631, Angew. Chem. Internat. Ed. Eng. 21: 567-583; Mata and Lobo, 1993, Tetrahedron: Asymmetry 4: 657-668) or can be characterized by the manner in which the molecule rotates the plane of polarized light and is designated dextrorotatory or levorotatory (namely, as (+)- or (−)-isomers, respectively). A chiral compound can exist as either an individual enantiomer or as a mixture thereof. A mixture containing equal proportions of enantiomers is called a “racemic mixture”.

In certain embodiments, the compounds disclosed herein can possess one or more asymmetric centers; and such compounds can therefore be produced as the individual (R)- or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, stereoisomers of the compounds provided herein are depicted upon treatment with base.

In certain embodiments, the compounds disclosed herein are “stereochemically pure”. A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

As used herein, the terms “subject” and “patient” are used interchangeably herein. The terms “subject” and “subjects” refer to a primate such as a monkey such as a cynomolgous monkey, a chimpanzee, and a human or non-primate animal. In one embodiment, the subject is a human. In another embodiment, the subject is a companion animal such as a dog or cat. In a further embodiment the subject is an animal of agricultural importance such as a sheep, cow, horse, goat, fish, pig, or domestic fowl (such as a chicken, turkey, duck, or goose).

In addition, a pharmaceutically acceptable prodrug of the compound represented by the formula (I) is also included in the present invention. The pharmaceutically acceptable prodrug refers to a compound having a group which can be converted into an amino group, a hydroxyl group, a carboxyl group, or the like, by solvolysis or under a physiological condition. Examples of the groups forming the prodrug include those as described in Prog. Med., 5, 2157-2161 (1985) or “Pharmaceutical Research and Development” (Hirokawa Publishing Company, 1990), vol. 7, Drug Design, 163-198. The term prodrug is used throughout the specification to describe any pharmaceutically acceptable form of a compound which, upon administration to a patient, provides the active compound. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound, Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The present invention includes all pharmaceutically acceptable isotopically-labelled compounds of the invention wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of the invention, such as those incorporating a radioactive isotope, may be useful in drug or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of the invention can generally be prepared by conventional techniques known to those skilled in the art or by processes analogous to those described in the accompanying Examples and Preparations using an appropriate isotopically-labeled reagents in place of the non-labeled reagent previously employed.

### Compounds

Isoxazoline derivatives have been disclosed in the art as having insecticidal and acaricidal activity. For example, WO2007/105814, WO2008/122375, and WO2009/035004 contain certain alkylene linked amides. WO2010/032437 discloses that the benzyl amide can be moved to the position ortho to the isoxazoline. Further, WO2007/075459 discloses phenyl isoxazolines substituted with 5- to 6-membered heterocycles, and WO2010/084067 and WO2010/025998 disclose phenyl isoxazolines substituted with 10- to 11-membered fused aryl and heteroaryls. Chiral processes for manufacturing isoxazolines have been reported in WO2011/104089 and WO2009/063910. Isoxazoline azetidine derivatives were published in WO2012/017359. Some spiro-azetidine isobenzofuran derivatives for the treatment of diabetes and hyperlipidemia were described in WO2008/096746. In addition, spirocyclic isoxazolines were recently published in WO2012/120399. WO2014/039489 discloses spirocyclic derivatives as antiparisitic agents, including azetidinyl-isobenzofurans, but the citation does not teach or suggest isothiazolines as the heterocyclic moiety. WO2014/079935 discloses a preparation of [4-(isothiazol-3-yl)arylthio]acetamide derivatives as insecticides, and WO2014/001121 and WO2014/001120 each disclose the preparation of isothiazole derivatives as insecticidal compounds, but none contain the azetidinyl-isobenzofuran. WO2014/206911 discloses isothiazoline compounds, however, the teaching lacks any azetidenyl-isobenzofuran moiety. WO2014/079941 discloses insecticidal compounds based on N-(arylsulfanylmethyl) carboxamide derivatives. US2014378415 discloses isothiazoline compounds, however, the teaching lacks any azetidenyl-isobenzofuran moiety. WO2009/112275 relates to pesticidal condensed-ring aryl compounds, however, the teaching lacks any azetidenyl-isobenzofuran moiety.

None of the foregoing references teach or suggest non-isoxazoline spirocyclic molecules, or processes of manufacturing such compounds. Nor do the foregoing citations indicate that such compounds would be useful against a spectrum of parasitic species relevant to companion animals, livestock, birds, or fish, and especially against the range of parasitic morphological lifecycle stages.

### Synthesis

Generally the compounds of the invention can be prepared, isolated or obtained by any method apparent to those of skill in the art. Exemplary methods of preparation are illustrated by the following schemes.

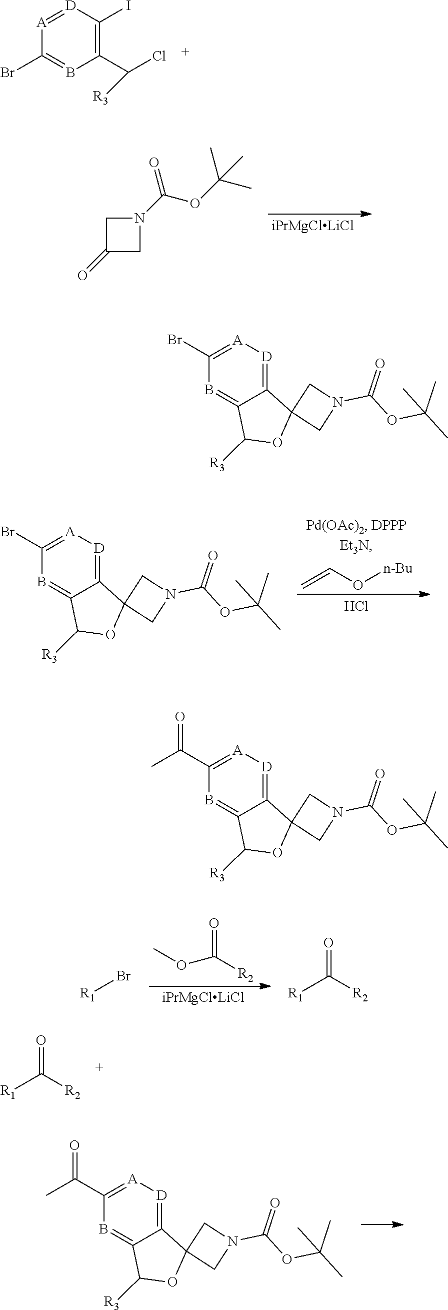

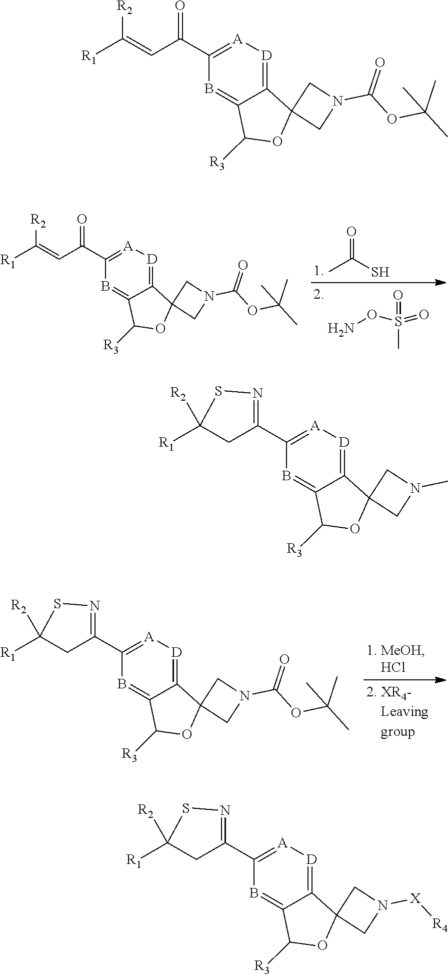

Alternatively, the isothiazoline can be formed according the below scheme:

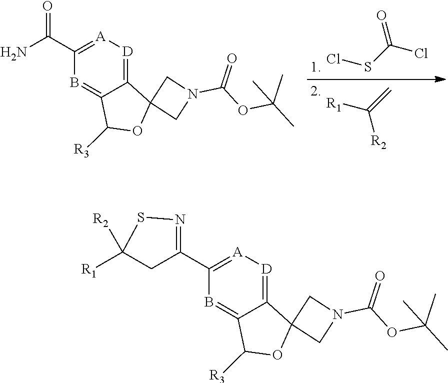

Formation of the key spirocyclic isobenzofuran bromide intermediate for analogs wherein A, B or D is N has been described in WO2014039484.

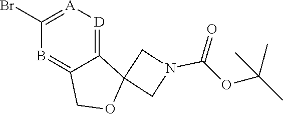

Key olefin intermediate for analogs wherein R2 is CH3 can be prepared according to the schemes below.

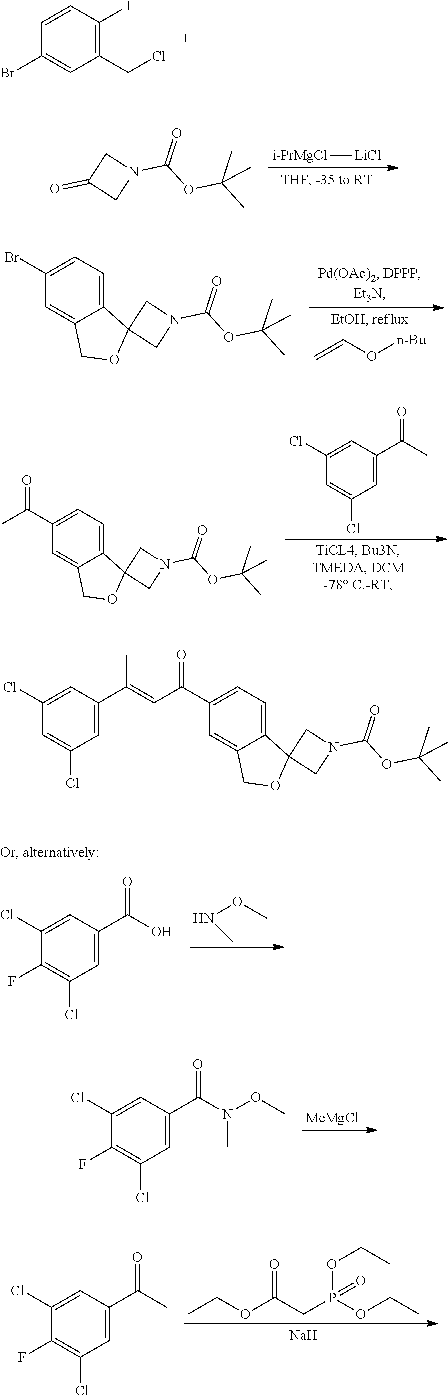

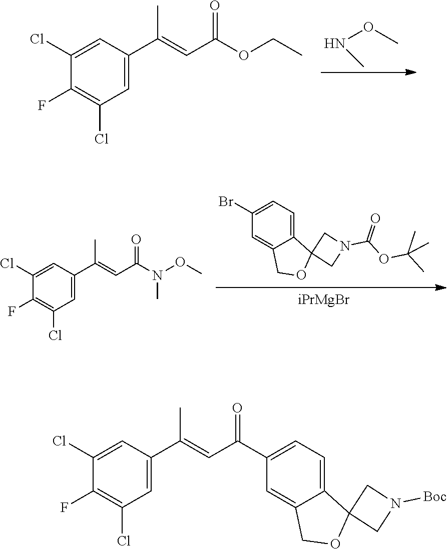

Analogs wherein R3 is other than hydrogen, for example where R3 is ═O, may be prepared according to the scheme below.

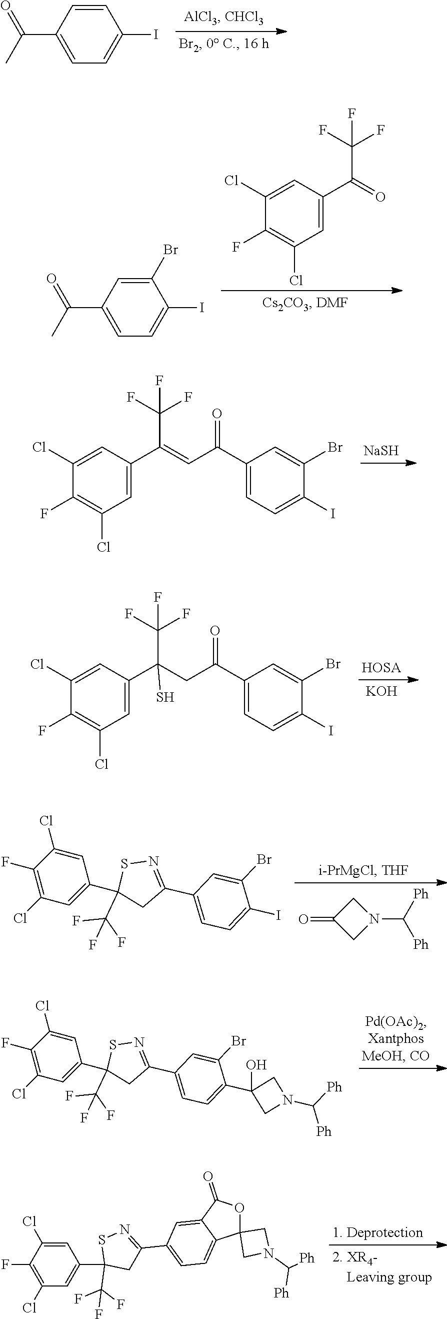

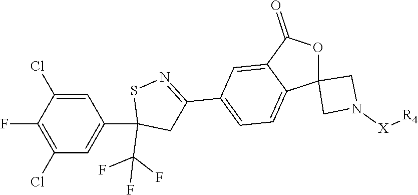

### Compositions and Methods of Administration

The compounds of formula (I) used in the methods disclosed herein can be administered in certain embodiments using veterinary, pharmaceutical, or pesticidal compositions including at least one compound of formula (I), if appropriate in the salt form, either used alone or in the form of a combination with one or more compatible and veterinarily, pharmaceutically, or pesticidally acceptable carriers, such as diluents or adjuvants, or with another agent. There are provided compositions which comprise an isothiazoline derivative of formula (I) or a salt thereof, and an acceptable excipient, carrier or diluent. The composition can also be in a variety of forms which include, but are not limited to, oral formulations, injectable formulations, and topical, dermal or subdermal formulations.

The composition can be in a form suitable for oral use, for example, as dietary supplements, troches, lozenges, chewables, tablets, hard or soft capsules, emulsions, aqueous or oily suspensions, aqueous or oily solutions, dispersible powders or granules, syrups, or elixirs. Compositions intended for oral use can be prepared according to any method known in the art for the manufacture of veterinary, pharmaceutical, or pesticidal compositions and such compositions can contain one or more agents selected from the group consisting of sweetening agents, bittering agents, flavoring agents, coloring agents and preserving agents in order to provide elegant and palatable preparations.

Tablets can contain the active ingredient in admixture with non-toxic, pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period.

Formulations for oral use can be hard gelatin capsules, wherein the active ingredient is mixed with an inert solid diluent, for example, calcium carbonate, calcium phosphate or kaolin. Capsules can also be soft gelatin capsules, wherein the active ingredient is mixed with water or miscible solvents such as propylene glycol, PEGs and ethanol, or an oil medium, for example, peanut oil, liquid paraffin, or olive oil.

The compositions can also be in the form of oil-in-water or water-in-oil emulsions. The oily phase can be a vegetable oil, for example, olive oil or arachis oil, or a mineral oil, for example, liquid paraffin or mixtures of these. Suitable emulsifying agents may be naturally-occurring phosphatides, for example, soy bean, lecithin, and esters or partial esters derived from fatty acids and hexitol anhydrides, for example, sorbitan monoleate, and condensation products of the said partial esters with ethylene oxide, for example, polyoxyethylene sorbitan monooleate. The emulsions can also contain sweetening agents, bittering agents, flavoring agents, and preservatives.

In one embodiment of the formulation, the composition is in the form of a microemulsion. Microemulsions are well suited as the liquid carrier vehicle. Microemulsions are quaternary systems comprising an aqueous phase, an oily phase, a surfactant and a cosurfactant. They are translucent and isotropic liquids. Microemulsions are composed of stable dispersions of microdroplets of the aqueous phase in the oily phase or conversely of microdroplets of the oily phase in the aqueous phase. The size of these microdroplets is less than 200 nm (1000 to 100,000 nm for emulsions). The interfacial film is composed of an alternation of surface-active (SA) and co-surface-active (Co-SA) molecules which, by lowering the interfacial tension, allows the microemulsion to be formed spontaneously. In one embodiment of the oily phase, the oily phase can be formed from mineral or vegetable oils, from unsaturated polyglycosylated glycerides or from triglycerides, or alternatively from mixtures of such compounds. In one embodiment of the oily phase, the oily phase comprises of triglycerides; in another embodiment of the oily phase, the triglycerides are medium-chain triglycerides, for example, C₈-C₁₀ caprylic/capric triglyceride. In another embodiment, the oily phase will represent a v/v range selected from the group consisting of about 2 to about 15%; about 7 to about 10%; and about 8 to about 9% v/v of the microemulsion. The aqueous phase includes, for example, water or glycol derivatives, such as propylene glycol, glycol ethers, polyethylene glycols or glycerol. In one embodiment of the glycol derivatives, the glycol is selected from the group consisting of propylene glycol, diethylene glycol monoethyl ether, dipropylene glycol monoethyl ether and mixtures thereof. Generally, the aqueous phase will represent a proportion from about 1 to about 4% v/v in the microemulsion. Surfactants for the microemulsion include diethylene glycol monoethyl ether, dipropylene glycol monomethyl ether, polyglycolyzed C₈-C₁₀ glycerides or polyglyceryl-6 dioleate. In addition to these surfactants, the cosurfactants include short-chain alcohols, such as ethanol and propanol. Some compounds are common to the three components discussed above, for example, aqueous phase, surfactant and cosurfactant. However, it is well within the skill level of the practitioner to use different compounds for each component of the same formulation. In one embodiment for the amount of surfactant/cosurfactant, the cosurfactant to surfactant ratio will be from about 1/7 to about ½.

In another embodiment for the amount of cosurfactant, there will be from about 25 to about 75% v/v of surfactant and from about 10 to about 55% v/v of cosurfactant in the microemulsion.

Oily suspensions can be formulated by suspending the active ingredient in a vegetable oil, for example, atachis oil, olive oil, sesame oil or coconut oil, or in mineral oil such as liquid paraffin. The oily suspensions can contain a thickening agent, for example, beeswax, hard paraffin or cetyl alcohol. Sweetening agents such as sucrose, saccharin or aspartame, bittering agents, and flavoring agents can be added to provide a palatable oral preparation. These compositions can be preserved by the addition of an anti-oxidant such as ascorbic acid, or other known preservatives.

Aqueous suspensions can contain the active material in admixture with excipients suitable for the manufacture of aqueous suspensions. Such excipients are suspending agents, for example, sodium carboxymethylcellulose, methylcellulose, hydroxypropylmethylcellulose, sodium alginate, polvinylpyrrolidone, gum tragacanth and gum acacia; dispersing or wetting agents can be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example, polyethylene sorbitan monooleate. The aqueous suspensions can also contain one or more preservatives, for example, ethyl, or n-propyl, p-hydroxybenzoate, one or more coloring agents, one or more flavoring agents, and one or more sweetening agents and/or bittering agents, such as those set forth above.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring and coloring agents, can also be present.

Syrups and elixirs can be formulated with sweetening agents, for example, glycerol, propylene glycol, sorbitol or sucrose. Such formulations may also contain a demulcent, a preservative, flavoring agent(s) and coloring agent(s).

The compositions can be in the form of a sterile injectable aqueous or oleagenous suspension. This suspension can be formulated according to the known art using those suitable dispersing or wetting agents and suspending agents which have been mentioned above. The sterile injectable preparation can also be a sterile injectable solution or suspension in a non-toxic parenterally-acceptable diluent or solvent, for example, as a solution in 1,3-butane diol. Among the acceptable vehicles and solvents that can be employed are water, Ringer's solution and isotonic sodium chloride solution. Cosolvents such as ethanol, propylene glycol or polyethylene glycols can also be used. Preservatives, such as phenol or benzyl alcohol, can be used.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Topical, dermal and subdermal formulations can include emulsions, creams, ointments, gels or pastes.

Organic solvents that can be used in the invention include but are not limited to: acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, or a mixture of at least two of these solvents.

As vehicle or diluent, compositions of the present invention may include plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈-C₁₂) triglycerides.

Dosage forms can contain from about 0.5 mg to about 5 g of an active agent.

In one embodiment of the invention, the active agent is present in the formulation at a concentration of about 0.05 to 10% weight/volume.

The compounds of formula (I) can be employed as such or in the form of their preparations or formulations as combinations with other pesticidally active substances, such as, for example, insecticides, attractants, sterilants, nematicides, acaricides, fungicides, herbicides, and with safeners, fertilizers and/or growth regulators.

The compounds of formula (I) according to the invention may be combined with one or more agents having the same sphere of activity, for example, to increase activity, or with substances having another sphere of activity, for example, to broaden the range of activity. The compounds of the present invention may also be combined with so-called repellents. By combining the compounds of the formula I with other suitable parasiticides, not only the parasiticidal activity can be enhanced, but the greatest part of those parasites that produce great economic damage will be covered. Moreover, this action will contribute substantially to avoiding the formation of resistance. Preferred groups of combination partners and especially preferred combination partners are named in the following, whereby combinations may contain one or more of these partners in addition to a compound of formula I. Suitable partners may also be afoxolaner, sarolaner, fluralaner, or a combination thereof. Any of the individually listed agents can be used in combination with compounds of formula (I) along with any other one or more listed agents independently.

Suitable partners in the mixture may be biocides, namely insecticides and acaricides with a varying mechanism of activity, for example, chitin synthesis inhibitors, growth regulators, active ingredients which act as juvenile hormones, active ingredients which act as adulticides, broadband insecticides, broadband acaricides and nematicides, and also anthelminthics and insect- and acarid-deterring substances, repellents or detachers. Non-limiting examples of suitable insecticides and acaricides are:

| | | |
|---|---|---|
| 1. Abamectin | 96. Dioxathion | 191. Omethoate |
| 2. Acephate | 97. Disulfoton | 192. Oxamyl |
| 3. Acequinocyl | 98. DNOC | 193. Oxydemethon M |
| 4. Acetamiprid | 99. Doramectin | 194. Oxydeprofos |
| 5. Acetoprole | 100. DPX-HGW86 | 195. Parathion |
| 6. Acrinathrin | 101. Edifenphos | 196. Parathion-methyl |
| 7. AKD-1022 | 102. Emamectin | 197. Permethrin |
| 8. Alanycarb | 103. Empenthrin | 198. Phenothrin |
| 9. Aldicarb | 104. Endosulfan | 199. Phenthoate |
| 10. Aldoxycarb | 105. Esfenvalerat | 200. Phorate |
| 11. Allethrin | 106. Ethiofencarb | 201. Phosalone |
| 12. Alpha-cypermethrin | 107. Ethion | 202. Phosmet |
| 13. Alphamethrin | 108. Ethiprole | 203. Phosphamidon |
| 14. Amidoflumet | 109. Ethoprophos | 204. Phoxim |
| 15. Amitraz | 110. Etofenprox | 205. Pirimicarb |
| 16. Anabasine | 111. Etoxazole | 206. Pirimiphos A |
| 17. Avermectin B1 | 112. Etrimphos | 207. Pirimiphos M |
| 18. Azadirachtin | 113. Fenamiphos | 208. Polynactins |
| 19. Azamethiphos | 114. Fenazaquin | 209. Prallethrin |
| 20. Azinphos-ethyl | 115. Fenbutatin oxide | 210. Profenofos |
| 21. Azinphos-methyl | 116. Fenitrothion | 211. Profluthrin |
| 22. Azocyclotin | 117. Fenobucarb | 212. Promecarb |
| 23. Bacillus subtil, toxin | 118. Fenothiocarb | 213. Propafos |
| 24. Bacillus thuringiensis | 119. Fenoxycarb | 214. Propargite |
| 25. Benclothiaz | 120. Fenpropathrin | 215. Propoxur |
| 26. Bendiocarb | 121. Fenpyroximate | 216. Prothiofos |
| 27. Benfuracarb | 122. Fenthion | 217. Prothoate |
| 28. Bensultap | 123. Fenvalerate | 218. Protrifenbute |
| 29. Benzoximate | 124. Fipronil | 219. Pymetrozine |
| 30. Beta-cyfluthrin | 125. Flonicamid | 220. Pyrachlofos |
| 31. Beta-cypermethrin | 126. Fluacrypyrim | 221. Pyrafluprole |
| 32. Bifenazate | 127. Fluazinam | 222. Pyresmethrin |
| 33. Bifenthrin | 128. Fluazuron | 223. Pyrethrin |
| 34. Bioallethrin | 129. Flubendiamide | 224. Pyrethrum |
| 35. Bioresmethrin | 130. Flucycloxuron | 225. Pyridaben |
| 36. Bistrifluron | 131. Flucythrinate | 226. Pyridalyl |
| 37. BPMC | 132. Flufenerim | 227. Pyridaphenthion |
| 38. Brofenprox | 133. Flufenoxuron | 228. Pyrifluquinazon |
| 39. Bromophos A | 134. Flufenprox | 229. Pyrimidifen |
| 40. Bromopropylate | 135. Flumethrin | 230. Pyriprole |
| 41. Bufencarb | 136. Fonophos | 231. Pyriproxyfen |
| 42. Buprofezin | 137. Formothion | 232. Quinalphos |
| 43. Butocarboxim | 138. Fosthiazate | 233. Resmethrin |
| 44. Cadusafos | 139. Fubfenprox | 234. Rotenone |
| 45. Carbaryl | 140. Furathiocarb | 235. RU 15525 |
| 46. Carbofuran | 141. Gamma-cyhalothrin | 236. Sabadilla |
| 47. Carbophenothion | 142. Halfenprox | 237. Salithion |
| 48. Carbosulfan | 143. Halofenozide | 238. Selamectin |
| 49. Cartap | 144. HCH | 239. Silafluofen |
| 50. Chloethocarb | 145. Heptenophos | 240. Spinetoram |
| 51. Chlorantraniliprole | 146. Hexaflumuron | 241. Spinosad |
| 52. Chlorethoxyfos | 147. Hexythiazox | 242. Spirodiclofen |
| 53. Chlorfenapyr | 148. Hydramethylnon | 243. Spiromesifen |
| 54. Chlorfenvinphos | 149. Hydroprene | 244. Spirotetramat |
| 55. Chlorfluazuron | 150. Imidacloprid | 245. Sulcofuron sodium |
| 56. Chlormephos | 151. Imiprothrin | 246. Sulfluramid |
| 57. Chlorpyrifos | 152. Indoxacarb | 247. Sulfotep |
| 58. Chlorpyrifos-methyl | 153. insect-active fungi | 248. Sulfur |
| 59. Chromafenozide | 154. insect-active nematodes | 249. Sulprofos |
| 60. Cis-Resmethrin | 155. insect-active viruses | 250. Tau-fluvalinate |
| 61. Clofentezin | 156. Iprobenfos | 251. Tebufenozide |
| 62. Clothianidin | 157. Lsofenphos | 252. Tebufenpyrad |
| 63. Coumaphos | 158. Isoprocarb | 253. Tebupirimfos |
| 64. Cyanophos | 159. Isoxathion | 254. Teflubenzuron |
| 65. Cycloprothrin | 160. Ivermectin | 255. Tefluthrin |
| 66. Cyenopyrafen | 161. Karanjin | 256. Temephos |
| 67. Cyflumetofen | 162. Kinoprene | 257. Terbufos |
| 68. Cyfluthrin | 163. Lamba-Cyhalothrin | 258. Tetrachlorvinphos |
| 69. Cyhalothrin | 164. Lepimectin | 259. Tetradifon |
| 70. Cyhexatin | 165. Lufenuron | 260. Tetramethrin |
| 71. Cymiazole | 166. Malathion | 261. Thiacloprid |
| 72. Cypermethrin | 167. Mecarbam | 262. Thiamethoxam |
| 73. Cyphenothrin | 168. Mesulfenphos | 263. Thiocyclam |
| 74. Cyromazine | 169. Metaflumizone | 264. Thiodicarb |
| 75. Deltamethrin | 170. Metaldehyde | 265. Thiofanox |
| 76. Demeton M | 171. Methamidophos | 266. Thionazin |
| 77. Demeton S | 172. Methidathion | 267. Thiosultap |
| 78. Demeton-S-methyl | 173. Methiocarb | 268. Thuringiensin |
| 79. Diafenthiuron | 174. Methomyl | 269. Tolfenpyrad |
| 80. Diazinon | 175. Methoprene | 270. Tralomethrin |
| 81. Dichlofenthion | 176. Methothrin | 271. Transfluthrin |
| 82. Dichlorvos | 177. Methoxyfenozide | 272. Triarathene |
| 83. Dicofol | 178. Metofluthrin | 273. Triazamate |
| 84. Dicrotophos | 179. Metolcarb | 274. Triazophos |
| 85. Dicyclanil | 180. Metoxadiazone | 275. Trichlorfon |
| 86. Diethion | 181. Mevinphos | 276. Triflumuron |
| 87. Diflovidazin | 182. Milbemectin | 277. Trimethacarb |
| 88. Diflubenzuron | 183. Milbemycin oxime | 278. Vamidothion |
| 89. Dimefluthrin | 184. Monocrotophos | 279. Vaniliprole |
| 90. Dimethoate | 185. Moxidectin | 280. XMC (3,5,-Xylylmethylcarbamate) |
| 91. Dimethylvinphos | 186. Naled | 281. Xylylcarb |
| 92. Dinobuton | 187. Nicotine | 282. Zeta-cypermethrin |
| 93. Dinocap | 188. Nitenpyram | 283. Zetamethrin |
| 94. Dinotefuran | 189. Novaluron | 284. ZXI 8901 |
| 95. Diofenolan | 190. Noviflumuron | 285. Demiditraz |
| 286. Afoxolaner | 287. Sarolaner | 288. Fluralaner |

Non-limitative examples of suitable anthelmintics, a few representatives have anthelmintic activity in addition to the insecticidal and acaricidal activity include:

| | | |
|---|---|---|
| (A1) Abamectin | (A2) Albendazole | (A3) Cambendazole |
| (A4) Closantel | (A5) Diethylcarbamazine | (A6) Doramectin |
| (A7) Emodepside | (A8) Eprinomectin | (A9) Febantel |
| (A10) Fendendazole | (A11) Flubendazole | (A12) Ivermectin |
| (A13) Levamisol | (A14) Mebendazole | (A15) Milbemectin |
| (A16) Milbemycin | (A17) Morantel | (A18) Moxidectin |
| Oxime | | |
| (A19) Nitroscanate | (A20) Omphalotin | (A21) Oxantel |
| (A22) Oxfendazole | (A23) Oxibendazole | (A24) Phenothiazine |
| (A25) Piperazine | (A26) PNU-97333 | (A27) PNU-141962 |
| (A28) Praziquantel | (A29) Pyrantel | (A30) Thiabendazole |

(A31) Triclabendazole amino acetonitrile derivatives named in
WO2005044784

Non-limitative examples of suitable repellents and detachers include:

(R1) DEET (N, N-diethyl-m-toluamide)
(R2) KBR 3023, picaridin, N-butyl-2-oxycarbonyl-(2-hydroxy)-piperidine
(R3) Cymiazole, N,-2,3-dihydro-3-methyl-1,3-thiazol-2-ylidene-2,4-xylidene

The above-specified combination partners are best known to specialists in this field. Most are described in various editions of the Pesticide Manual, The British Crop Protection Council, London, in various editions of the Compendium of Veterinary Products, North American Compendiums, Inc., in various editions of the Compendium of Pesticide Common Names and in various editions of the Merck Veterinary Manual and The Merck Index, Merck & Co., Inc., Rahway, N.J., USA.

The pharmaceutical preparation comprising the isothiazoline derivatives, for delivery to a human or other mammal, is preferably in unit dosage form, in which the preparation is subdivided into unit doses containing an appropriate quantity of the active component. The unit dosage form can be a packaged preparation containing discrete quantities of the preparation, such as packaged tablets, capsules, and powders in vials or ampoules. Also, the unit dosage form can be a capsule, tablet or lozenge itself, or it can be an appropriate number of any of these in packaged form.

The quantity of active component in a unit dose preparation can be varied or adjusted from about 0.1 mg to about 1000 mg, according to the particular application and the potency of the active component. The composition can, if desired, also contain other compatible therapeutic agents.

In therapeutic use for the treatment or prevention of a parasitic infection in a human or other mammal, the compounds utilized in the method of treatment are administered at an initial dosage of about 0.1 mg/kg to about 100 mg/kg per interval. Preferred intervals may be daily, weekly, monthly, quarterly, semi-annually, or annually. The dosages can be varied depending on the requirements of the patient, for example, the size of the human or mammal being treated, the severity of the condition being treated, the route of administration, and the potency of the compound(s) being used. Determination of the proper dosage and route of administration for a particular situation is within the skill of the practitioner. Generally, the treatment will be initiated with smaller dosages which are less than the optimum dose of the compound, which can be increased in small increments until the optimum effect under the particular circumstances of the infection is reached. For convenience, the total daily dosage can be divided and administered in portions during the day if desired.

The compounds of the present invention, stereoisomers thereof, and compositions comprising a therapeutically effective amount of a Formula (I) compound, stereoisomer thereof, and veterinarily acceptable salt thereof, and a veterinarily acceptable excipient, diluent, or carrier are useful as ectoparasiticides for the control and treatment of infections or infestations manifested by said ectoparasite in an animal. The compounds of the present invention are illustrated herein to have utility as an ectoparasiticide, in particular, as an acaricide and insecticide. They may, in particular, be used in the fields of veterinary medicine, livestock husbandry and the maintenance of public health: against acarids, insects, and copepods which are parasitic upon vertebrates, particularly warm-blooded vertebrates, including companion animals, livestock, and fowl and cold-blooded vertebrates like fish. Non-limiting examples of ectoparasites include: ticks (e.g., Ixodes spp., (e.g., I. ricinus, I. hexagonus), Rhipicephalus spp. (e.g., R. sanguineus), Boophilus spp., Amblyomma spp. (e.g., A. americanum, A. maculatum, A. triste, A. parvum, A. cajennense, A. ovate, A. oblongoguttatum, A. aureolatum, A. cajennense), Hyalomma spp., Haemaphysalis spp., Dermacentor spp. (e.g., D. variabilis, D. andersoni, D. marginatus), Ornithodorus spp., and the like); mites (e.g., Dermanyssus spp., Sarcoptes spp. (e.g., S. scabiei), Psoroptes spp. (e.g., P. bovis), Otodectes spp., Chorioptes spp., Demodex spp., (e.g., D. folliculorum, D. canis, and D. brevis) and the like); chewing and sucking lice (e.g., Damalinia spp., Linognathus spp., Cheyletiella spp., Haematopinus spp., Solenoptes spp., Trichodectes spp., Felicola spp., and the like); fleas (e.g., Siphonaptera spp., Ctenocephalides spp., and the like); biting flies, midges, and mosquitos (e.g., Tabanidae spp., Haematobia spp., Musca spp., Stomoxys spp., Dematobia spp., Cochliomyia spp., Simuliidae spp., Ceratopogonidae spp., Psychodidae spp., Aedes spp., Culex spp., Anopheles spp., Lucilia spp., Phlebotomus spp., Lutzomyia spp., and the like); bed bugs (e.g., insects within the genus Cimex and family Cimicidae); and grubs (e.g., Hypoderma bovis, H. lineatum); and copepods (e.g., sea lice within the Order Siphonostomatoida, including genera Lepeophtheirus and Caligus).

The compound of the present invention can also be used for the treatment of endoparasites, for example, helminths (e.g., trematodes, cestodes, and nematodes) including heartworm, roundworm, hookworm, whipworm, fluke, and tapeworm. The gastrointestinal roundworms include, for example, Ostertagia ostertagi (including inhibited larvae), O. lyrata, Haemonchus placei, H. similis, H. contortus, Toxocara canis, T. leonina, T. cati, Trichostrongylus axei, T. colubriformis, T. longispicularis, Cooperia oncophora, C. pectinata, C. punctata, C. surnabada (syn. mcmasteri), C. spatula, Ascaris suum, Hyostrongylus rubidus, Bunostomum phlebotomum, Capillaria bovis, B. trigonocephalum, Strongyloides papillosus, S. ransomi, Oesophagostomum radiatum, O. dentatum, O. columbianum, O. quadrispinulatum, Trichuris spp., and the like. Other parasites include: hookworms (e.g., Ancylostoma caninum, A. tubaeforme, A. braziliense, Uncinaria stenocephala); lungworms (e.g., Dictyocaulus viviparus and Metastrongylus spp); eyeworms (e.g., Thelazia spp.); parasitic stage grubs (e.g., Hypoderma bovis, H. lineatum, Dermatobia hominis); kidneyworms (e.g., Stephanurus dentatus); screw worm (e.g., Cochliomyia hominivorax (larvae); filarial nematodes of the super-family Filarioidea and the Onchocercidae Family. Non-limiting examples of filarial nematodes within the Onchocercidae Family include the genus Brugia spp. (i.e., B. malayi, B. pahangi, B. timori, and the like), Wuchereria spp. (i.e., W. bancrofti, and the like), Dirofilaria spp. (D. immitis, D. repens, D. ursi, D. tenuis, D. spectans, D. lutrae, and the like), Dipetalonema spp. (i.e., D reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. boa).

Preferably, the compounds of the present invention are used to treat parasitic infection or infestation, preferably wherein the parasite is a flea or tick. In particularly preferred embodiments, the parasite is C. felis, R. sanguineis, A. americanum, I. scapularis, A. maculate, D. variabilis, or I. ricinus.

In another aspect of the invention, the compound of the present invention is useful for treating endoparasiticidal infection from helminths/filarial nematodes within the genus Dirofilaria (i.e., D. immitis, D. repens, D. ursi, D. tenuis, and the like).

In another aspect of the invention, the compounds of the present invention are used to prevent transmission of disease from biting insects, such as mosquitoes (e.g., Tabanidae spp., Haematobia spp., Musca spp., Stomoxys spp., Dematobia spp., Cochliomyia spp., Simuliidae spp., Ceratopogonidae spp., Psychodidae spp., Aedes spp., Culex spp., Anopheles spp., Lucille spp., Phlebotomus spp., Lutzomyia spp., and the like).

The compounds of the present invention, stereoisomers thereof, and veterinarily or pharmaceutically acceptable salts thereof, and compositions comprising compounds of the present invention in conjunction with at least one other veterinary agent are of particular value in the control of ectoparasites, endoparasites, and insects which are injurious to, or spread or act as vectors of diseases in companion animals, livestock, birds, and fish.

Any of the compounds of the present invention, or a suitable combination of a compound of the present invention and optionally, with at least one additional veterinary agent may be administered directly to the animal and/or indirectly by applying it to the local environment in which the animal dwells (such as bedding, enclosures, and the like). Direct administration includes contacting the skin, fur, or feathers of a subject animal with the compound(s), or by feeding or injecting the compounds into the animal.

The Formula (I) compound, stereoisomer thereof, and veterinarily acceptable salt thereof, and combinations with at least one additional veterinary agent, as described herein, are believed to be of value for the treatment and control of the various lifecycle stages of insects and parasites including egg, nymph, larvae, juvenile and adult stages.

The present invention also relates to a method of administering a compound of the present invention alone or in combination with at least one additional veterinary agent, and optionally a veterinarily acceptable excipient, diluent, or carrier, to animals in good health comprising the application to said animal to reduce or eliminate the potential for human parasitic infection or infestation from parasites carried by the animal and to improve the environment in which the animals inhabit.

The present invention explicitly encompasses those compounds presented in Table 1. A composition comprising a therapeutically acceptable amount of any of these compounds is also within the scope of the invention. The composition can further comprise a veterinarily acceptable excipient, diluent, carrier, or mixture thereof. Such a composition can be administered to an animal in need thereof to treat and/or prevent a parasitic infection or infestation. The composition can further comprise an additional veterinary agent, as described herein.

TABLE 1

| Ref. | |
|---|---|
| No. | Compound Name |
| 1 | 1-[6-[5-(6-chloro-3-pyridyl)-5-(trifluoromethyl)-4H-isothiazol-3-yl]spiro[1H- |
| | isobenzofuran-3,3′-azetidine]-1′-yl]-3,3,3-trifluoro-propan-1-one |
| 2 | 1-[6-[5-(6-chloro-3-pyridyl)-5-(trifluoromethyl)-4H-isothiazol-3-yl]spiro[1H- |
| | isobenzofuran-3,3′-azetidine]-1′-yl]-2-methylsulfonyl-ethanone |
| 3 | 1-[6-[5-(6-chloro-3-pyridyl)-5-(trifluoromethyl)-4H-isothiazol-3-yl]spiro[1H- |
| | isobenzofuran-3,3′-azetidine]-1′-yl]-2-methyl-propan-1-one |
| 4 | 6-[5-(6-chloro-3-pyridyl)-5-(trifluoromethyl)-4H-isothiazol-3-yl]-N-ethyl- |
| | spiro[1H-isobenzofuran-3,3′-azetidine]-1′-carboxamide |
| 5 | 6-[5-(6-chloro-3-pyridyl)-5-(trifluoromethyl)-4H-isothiazol-3-yl]-1′- |
| | ethylsulfonyl-spiro[1H-isobenzofuran-3,3′-azetidine] |
| 6 | 1-[6-[5-(6-Chloro-2-pyridyl)-5-(trifluoromethyl)-4H-isothiazol-3-yl]spiro[1H- |
| | isobenzofuran-3,3′-azetidine]-1′-yl]-3,3,3-trifluoropropan-1-one |
| 7 | 1-[6-[5-(6-Chloro-2-pyridyl)-5-(trifluoromethyl)-4H-isothiazol-3-yl]spiro[1H- |
| | isobenzofuran-3,3′-azetidine]-1′-yl]-2-methylsulfonylethanone |
| 8 | 1-[6-[5-(6-Chloro-2-pyridyl)-5-(trifluoromethyl)-4H-isothiazol-3-yl]spiro[1H- |
| | isobenzofuran-3,3′-azetidine]-1′-yl]-2-methylpropan-1-one |
| 9 | 6-[5-(6-Chloro-2-pyridyl)-5-(trifluoromethyl)-4H-isothiazol-3-yl]-N- |
| | ethylspiro[1H-isobenzofuran-3,3′-azetidine]-1′-carboxamide |
| 10 | 6-[5-(6-Chloro-2-pyridyl)-5-(trifluoromethyl)-4H-isothiazol-3-yl]-1′- |
| | ethylsulfonylspiro[1H-isobenzofuran-3,3′-azetidine] |
| 11 | 6-[5-(3,5-dichlorophenyl)-5-(trifluoromethyl)-4H-isothiazol-3-yl]-N-ethyl- |
| | spiro[1H-isobenzofuran-3,3′-azetidine]-1′-carboxamide |
| 12 | 1-[6-[5-(3,5-dichlorophenyl)-5-(trifluoromethyl)-4H-isothiazol-3-yl]spiro[1H- |
| | isobenzofuran-3,3′-azetidine]-1′-yl]-3,3,3-trifluoro-propan-1-one |
| 13 | 1-[6-[5-(3,5-dichlorophenyl)-5-(trifluoromethyl)-4H-isothiazol-3-yl]spiro[1H- |
| | isobenzofuran-3,3′-azetidine]-1′-yl]-2-methyl-propan-1-one |
| 14 | 1-[6-[5-(3,5-dichlorophenyl)-5-(trifluoromethyl)-4H-isothiazol-3-yl]spiro[1H- |
| | isobenzofuran-3,3′-azetidine]-1′-yl]-2-methylsulfonyl-ethanone |
| 15 | 6-[5-(3,5-dichlorophenyl)-5-(trifluoromethyl)-4H-isothiazol-3-yl]-1′- |
| | ethylsulfonyl-spiro[1H-isobenzofuran-3,3′-azetidine] |
| 16 | 1-[6-[5-(3,5-dichloro-4-fluoro-phenyl)-5-(trifluoromethyl)-4H-isothiazol-3- |
| | yl]spiro[1H-furo[3,4-c]pyridine-3,3′-azetidine]-1′-yl]-3,3,3-trifluoro-propan-1- |
| | one |
| 17 | 1-[6-[5-(3,5-dichloro-4-fluoro-phenyl)-5-(trifluoromethyl)-4H-isothiazol-3- |
| | yl]spiro[1H-furo[3,4-c]pyridine-3,3′-azetidine]-1′-yl]-2-methyl-propan-1-one |
| 18 | 1-[6-[5-(3,5-dichloro-4-fluoro-phenyl)-5-(trifluoromethyl)-4H-isothiazol-3- |
| | yl]spiro[1H-furo[3,4-c]pyridine-3,3′-azetidine]-1′-yl]-2-methylsulfonyl- |
| | ethanone |
| 19 | 6-[5-(3,5-dichloro-4-fluoro-phenyl)-5-(trifluoromethyl)-4H-isothiazol-3-yl]-1′- |
| | ethylsulfonyl-spiro[1H-furo[3,4-c]pyridine-3,3′-azetidine] |
| 20 | 6-[5-(3,5-dichloro-4-fluoro-phenyl)-5-(trifluoromethyl)-4H-isothiazol-3-yl]-N- |
| | ethyl-spiro[1H-furo[3,4-c]pyridine-3,3′-azetidine]-1′-carboxamide |
| 21 | 1-[2-[5-(3,5-dichloro-4-fluoro-phenyl)-5-(trifluoromethyl)-4H-isothiazol-3- |
| | yl]spiro[7H-furo[3,4-b]pyridine-5,3′-azetidine]-1′-yl]-3,3,3-trifluoro-propan-1- |
| | one |
| 22 | 1-[2-[5-(3,5-dichloro-4-fluoro-phenyl)-5-(trifluoromethyl)-4H-isothiazol-3- |
| | yl]spiro[7H-furo[3,4-b]pyridine-5,3′-azetidine]-1′-yl]-2-methyl-propan-1-one |
| 23 | 1-[2-[5-(3,5-dichloro-4-fluoro-phenyl)-5-(trifluoromethyl)-4H-isothiazol-3- |
| | yl]spiro[7H-furo[3,4-b]pyridine-5,3′-azetidine]-1′-yl]-2-methylsulfonyl- |
| | ethanone |
| 24 | 2-[5-(3,5-dichloro-4-fluoro-phenyl)-5-(trifluoromethyl)-4H-isothiazol-3-yl]-1′- |
| | ethylsulfonyl-spiro[7H-furo[3,4-b]pyridine-5,3′-azetidine] |
| 25 | 2-[5-(3,5-dichloro-4-fluoro-phenyl)-5-(trifluoromethyl)-4H-isothiazol-3-yl]-N- |
| | ethyl-spiro[7H-furo[3,4-b]pyridine-5,3′-azetidine]-1′-carboxamide |
| 26 | 1-[3-[5-(3,5-dichloro-4-fluoro-phenyl)-5-(trifluoromethyl)-4H-isothiazol-3- |
| | yl]spiro[5H-furo[3,4-b]pyridine-7,3′-azetidine]-1′-yl]-3,3,3-trifluoro-propan-1- |
| | one |
| 27 | 1-[3-[5-(3,5-dichloro-4-fluoro-phenyl)-5-(trifluoromethyl)-4H-isothiazol-3- |
| | yl]spiro[5H-furo[3,4-b]pyridine-7,3′-azetidine]-1′-yl]-2-methyl-propan-1-one |
| 28 | 1-[3-[5-(3,5-dichloro-4-fluoro-phenyl)-5-(trifluoromethyl)-4H-isothiazol-3- |
| | yl]spiro[5H-furo[3,4-b]pyridine-7,3′-azetidine]-1′-yl]-2-methylsulfonyl- |
| | ethanone |
| 29 | 3-[5-(3,5-dichloro-4-fluoro-phenyl)-5-(trifluoromethyl)-4H-isothiazol-3-yl]-1′- |
| | ethylsulfonyl-spiro[5H-furo[3,4-b]pyridine-7,3′-azetidine] |
| 30 | 3-[5-(3,5-dichloro-4-fluoro-phenyl)-5-(trifluoromethyl)-4H-isothiazol-3-yl]-N- |
| | ethyl-spiro[5H-furo[3,4-b]pyridine-7,3′-azetidine]-1′-carboxamide |
| 31 | 1-[6-[5-(2,6-dichloro-4-pyridyl)-5-(trifluoromethyl)-4H-isothiazol-3- |
| | yl]spiro[1H-isobenzofuran-3,3′-azetidine]-1′-yl]-3,3,3-trifluoro-propan-1-one |
| 32 | 1-[6-[5-(2,6-dichloro-4-pyridyl)-5-(trifluoromethyl)-4H-isothiazol-3- |
| | yl]spiro[1H-isobenzofuran-3,3′-azetidine]-1′-yl]-2-methylsulfonyl-ethanone |
| 33 | 1-[6-[5-(2,6-dichloro-4-pyridyl)-5-(trifluoromethyl)-4H-isothiazol-3- |
| | yl]spiro[1H-isobenzofuran-3,3′-azetidine]-1′-yl]-2-methyl-propan-1-one |
| 34 | 6-[5-(2,6-dichloro-4-pyridyl)-5-(trifluoromethyl)-4H-isothiazol-3-yl]-N-ethyl- |
| | spiro[1H-isobenzofuran-3,3′-azetidine]-1′-carboxamide |
| 35 | 6-[5-(2,6-dichloro-4-pyridyl)-5-(trifluoromethyl)-4H-isothiazol-3-yl]-1′- |
| | ethylsulfonyl-spiro[1H-isobenzofuran-3,3′-azetidine] |
| 36 | 1-[6-[5-(3,5-dichlorophenyl)-5-methyl-4H-isothiazol-3-yl]spiro[1H- |
| | isobenzofuran-3,3′-azetidine]-1′-yl]-3,3,3-trifluoro-propan-1-one |
| 37 | 1-[6-[5-(3,5-dichlorophenyl)-5-methyl-4H-isothiazol-3-yl]spiro[1H- |
| | isobenzofuran-3,3′-azetidine]-1′-yl]-2-methyl-propan-1-one |
| 38 | 1-[6-[5-(3,5-dichlorophenyl)-5-methyl-4H-isothiazol-3-yl]spiro[1H- |
| | isobenzofuran-3,3′-azetidine]-1′-yl]-2-methylsulfonyl-ethanone |
| 39 | 6-[5-(3,5-dichlorophenyl)-5-methyl-4H-isothiazol-3-yl]-1′-ethylsulfonyl- |
| | spiro[1H-isobenzofuran-3,3′-azetidine] |
| 40 | 6-[5-(3,5-dichlorophenyl)-5-methyl-4H-isothiazol-3-yl]-N-ethyl-spiro[1H- |
| | isobenzofuran-3,3′-azetidine]-1′-carboxamide |
| 41 | 1-[6-[5-(3,5-dichloro-4-fluoro-phenyl)-5-methyl-4H-isothiazol-3-yl]spiro[1H- |
| | isobenzofuran-3,3′-azetidine]-1′-yl]-3,3,3-trifluoro-propan-1-one |
| 42 | 1-[6-[5-(3,5-dichloro-4-fluoro-phenyl)-5-methyl-4H-isothiazol-3-yl]spiro[1H- |
| | isobenzofuran-3,3′-azetidine]-1′-yl]-2-methyl-propan-1-one |
| 43 | 1-[6-[5-(3,5-dichloro-4-fluoro-phenyl)-5-methyl-4H-isothiazol-3-yl]spiro[1H- |
| | isobenzofuran-3,3′-azetidine]-1′-yl]-2-methylsulfonyl-ethanone |
| 44 | 6-[5-(3,5-dichloro-4-fluoro-phenyl)-5-methyl-4H-isothiazol-3-yl]-1′- |
| | ethylsulfonyl-spiro[1H-isobenzofuran-3,3′-azetidine] |
| 45 | 6-[5-(3,5-dichloro-4-fluoro-phenyl)-5-methyl-4H-isothiazol-3-yl]-N-ethyl- |
| | spiro[1H-isobenzofuran-3,3′-azetidine]-1′-carboxamide |
| 46 | 6′-[5-(3,5-dichloro-4-fluoro-phenyl)-5-(trifluoromethyl)-4H-isothiazol-3-yl]-1- |
| | (3,3,3-trifluoropropanoyl)spiro[azetidine-3,3′-isobenzofuran]-1′-one |
| 47 | 6′-[5-(3,5-dichloro-4-fluoro-phenyl)-5-(trifluoromethyl)-4H-isothiazol-3-yl]-1- |
| | (2-methylpropanoyl)spiro[azetidine-3,3′-isobenzofuran]-1′-one |
| 48 | 6′-[5-(3,5-dichloro-4-fluoro-phenyl)-5-(trifluoromethyl)-4H-isothiazol-3-yl]-1- |
| | (2-methylsulfonylacetyl)spiro[azetidine-3,3′-isobenzofuran]-1′-one |
| 49 | 6′-[5-(3,5-dichloro-4-fluoro-phenyl)-5-(trifluoromethyl)-4H-isothiazol-3-yl]-1- |
| | ethylsulfonyl-spiro[azetidine-3,3′-isobenzofuran]-1′-one |
| 50 | 5′-[5-(3,5-dichloro-4-fluoro-phenyl)-5-(trifluoromethyl)-4H-isothiazol-3-yl]-N- |
| | ethyl-3′-oxo-spiro[azetidine-3,1′-isobenzofuran]-1-carboxamide |

### Experimental Procedures:

Synthesis

The following Examples illustrate the synthesis of representative compounds of formula (I). These examples are not intended, nor are they to be construed, as limiting the scope of the embodiments disclosed herein. It will be clear that various embodiments may be practiced otherwise than as particularly described herein. Numerous modifications and variations are possible in view of the teachings herein and, therefore, are within the scope.

Liquid chromatography—mass spectrometry (LCMS) experiments to determine retention times and associated mass ions were performed using one or more of the following Methods A, B, and C:

Method A: Waters BEH C18, 3.0×30 mm, 1.7 μm, was used at a temperature of 50° C. and at a flow rate of 1.5 mL/min, 2 μL injection, mobile phase: (A) water with 0.1% formic acid and 1% acetonitrile, mobile phase (B) MeOH with 0.1% formic acid; retention time given in minutes.

Method A details: (I) ran on a Binary Pump G1312B with UV/Vis diode array detector G1315C and Agilent 6130 mass spectrometer in positive and negative ion electrospray mode with UV PDA detection with a gradient of 15-95% (B) in a 2.2 min linear gradient (II) hold for 0.8 min at 95% (B) (III) decrease from 95-15% (B) in a 0.1 min linear gradient (IV) hold for 0.29 min at 15% (B);

Method B: An Agilent Zorbax Bonus RP, 2.1×50 mm, 3.5 μm, was used at a temperature of 50 ° C. and at a flow rate of 0.8 mL/min, 2 μL injection, mobile phase: (A) water with 0.1% formic acid and 1% acetonitrile, mobile phase (B) MeOH with 0.1% formic acid; retention time given in minutes.

Method B details: (I) ran on a Binary Pump G1312B with UV/Vis diode array detector G1315C and Agilent 6130 mass spectrometer in positive and negative ion electrospray mode with UV-detection at 220 and 254 nm with a gradient of 5-95% (B) in a 2.5 min linear gradient (II) hold for 0.5 min at 95% (B) (III) decrease from 95-5% (B) in a 0.1 min linear gradient (IV) hold for 0.29 min at 5% (B).

Method C: An API 150EX mass spectrometer linked to a Shimadzu LC-10AT LC system with a diode array detector was used. The spectrometer had an electrospray source operating in positive and negative ion mode. LC was carried out using an Agilent ZORBAX XDB 50×2.1 mm C18 column and a 0.5 mL/minute flow rate. Solvent A: 95% water, 5% acetonitrile containing 0.01% formic acid; Solvent B: acetonitrile. The gradient was shown as below. 0-0.5 min: 2% solvent (B); 0.5-2.5 min: 2% solvent B to 95% solvent (B); 2.5-4.0 min: 95% solvent (B); 4.0-4.2 min: 95% solvent (B) to 2% solvent B; 4.2-6.0 min: 2% solvent (B).

### EXAMPLES

The following Examples provide a more detailed description of the process conditions for preparing compounds of the present invention. It is to be understood, however, that the invention, as fully described herein and as recited in the claims, is not intended to be limited by the details of the following schemes or modes of preparation.

### Example 1

1-(6-chloro-3-pyridyl)-2,2,2-trifluoro-ethanone

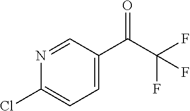 [SMILES: O=C(c1ccc(Cl)nc1)C(F)(F)F]

A round bottom flask equipped with a stir bar and nitrogen inlet was charged with the methyl 6-chloropyridine-3-carboxylate (21.9 g, 128 mmol), dimethoxyethane (64 mL) and catalytic CsF (195 mg, 1.28 mmol, 1 mol %). Once the material had dissolved, trimethyl(trifluoromethyl)silane (21 mL, 128 mmol) was added dropwise over 15 minutes. The reaction was allowed 3 h at RT and then 4 N HCl (94 mL) was added. The mixture was stirred vigorously overnight. The mixture was diluted with water (200 mL), then extracted with EtOAc (3×200 mL), the combined organic phases washed with saturated sodium bicarbonate (200 mL), dried (Na₂SO₄) and the solvent was evaporated under reduced pressure. The material was dissolved in benzene (100 mL) and the mixture was dehydrated using Dean-Stark apparatus for 18 h. It was cooled and the solvent was evaporated under reduced pressure. The material was purified by vacuum distillation using a vacuum of 0.1 mmHg, 70° C. bath temperature and collected the distillate fraction between 39° and 45° C. head temperature. This gave 1-(6-chloro-3-pyridyl)-2,2,2-trifluoro-ethanone which became a white solid on standing. Amount obtained : 15 g, 72 mmol, 56% yield. ES LC-MS m/z=242 (M+MeOH+H+). 1H NMR (CHLOROFORM-d) δ: 9.02-9.11 (m, 1H), 8.30 (ddt, J=8.5, 1.7, 0.7 Hz, 1H), 7.51-7.60 (m, 1H).
tert-Butyl 6-bromospiro[1H-isobenzofuran-3,3′-azetidine]-1′-carboxylate

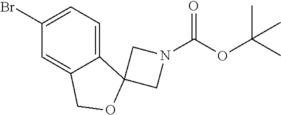 [SMILES: CC(C)(C)OC(=O)N1CC2(C1)OCc1cc(Br)ccc12]

In a 2 L 3-neck round bottomed flash equipped with two 250 mL addition funnels, a large stir bar and a N2 inlet adaptor was added 4-bromo-2-(chloromethyl)-1-iodo-benzene (50 g, 150.89 mmol) and THF (400 mL) was cannulated into the flask. The mixture was stirred under N2 until completely dissolved, then cooled between −35 and −40° C. i-PrMgCl—LiCl (121 mL, 156.77 mmol, 1.3 M in THF) was added dropwise while keeping the cold bath temperature between −33° C. and −36° C. The reaction was stirred within this temperature range for 1.5 h. A solution of tert-butyl 3-oxoazetidine-1-carboxylate (30.46 g, 178.04 mmol) in THF (120 mL) was added dropwise while maintaining the cold bath temperature range. The reaction was slowly warmed to room temperature over 2 h (cold bath removed) and then stirred at this temperature for 16 h. The reaction was slowly quenched with aqueous citric acid (1M, 300 mL), diluted with methyl-tertbutyl ether (MTBE) (300 mL), mixed and the layers separated. The aqueous phase was further extracted with MTBE (1×150 mL) and the combined organic layers was washed with saturated NaHCO3 (1×100 mL), brine (1×100 mL), dried (Na₂SO₄), filtered and evaporated to an orange oil. The oil was dissolved in EtOH (250 mL), the solution diluted with water (100 mL) and stirred at RT overnight. The resulting ppt was filtered, dried under vacuum at 50° C. to give 32.6 g. The filtrate was concentrated and subjected to chromatographic purification (0-15% Et0Ac/Heptanes) to give an additional 11.1 g fora combined yield of 43.7 g (85.2%) of a white solid. 1H NMR (400 MHz, CDCl3) δ: 7.49-7.53 (m, 1H), 7.36-7.38 (m, 1H), 7.35 (d, J=8.2 Hz, 1H), 5.09 (s, 2H), 4.29-4.33 (m, 2H), 4.11 (d, J=10.2 Hz, 2H), 1.49 (s, 9H); LCMS-ELSD (M+H) 341.2.
tert-Butyl 6-acetylspiro[1H-isobenzofuran-3,3′-azetidine]-1′-carboxylate

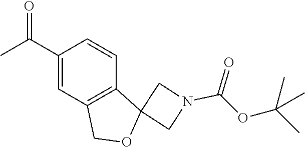 [SMILES: CC(=O)c1ccc2c(c1)COC21CN(C(=O)OC(C)(C)C)C1]

To a 70 mL glass pressure reaction tube was added a solution of tert-butyl 6-bromospiro[1H-isobenzofuran-3,3′-azetidine]-1′-carboxylate (5.0 g, 14.7 mmol) in EtOH (20 mL). Dry N2 was bubbled through the solution for 30 minutes. To this was added 1,3-bis(diphenylphosphino)propane (dppp) (364 mg, 0.88 mmol), Pd(OAc)2 (99 mg, 0.44 mmol), followed by triethylamine (3.7 mL, 26.45 mmol) and butyl vinyl ether (3.8 mL, 29.39 mmol). The vessel was sealed and placed in a 96° C. oil bath behind a blast shield and stirred at this temperature overnight. Upon cooling, the reaction was slowly quenched with 1N HCl (20 mL) to pH 2-3, then stirred at RT for 2 h. The pH of the reaction mixture was adjusted to pH 7 with saturated NaHCO3 solution and extracted with EtOAc (3×100 mL), washed with water, brine, dried (Na₂SO₄), filtered and evaporated. Purification by chromatography on silica gel with 0-50% EtOAc/Heptanes provided the product as a white solid. Yield 3.42 g (76.7%). 1H NMR (400 MHz, CDCl3) δ: 7.99 (d, J=8.0 Hz, 1H), 7.82 (s, 1H), 7.57 (d, J=7.9 Hz, 1H), 5.16 (s, 2H), 4.34 (d, J=9.4 Hz, 2H), 4.15 (d, J=9.2 Hz, 2H), 2.63 (s, 3H), 1.49 (s, 9H); LCMS-ELSD (M+H-isobutylene) 248.6. Ref: WO2014/039489.
tert-butyl 6-[(Z)-3-(6-chloro-3-pyridyl)-4,4,4-trifluoro-but-2-enoyl]spiro[1H-isobenzofuran-3,3′-azetidine]-1′-carboxylate

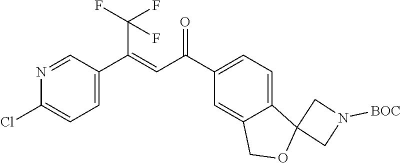 [SMILES: CC(C)(C)OC(=O)N1CC2(C1)OCc1cc(C(=O)/C=C(/c3ccc(Cl)nc3)C(F)(F)F)ccc12]

A round bottom flask equipped with a stir bar and nitrogen inlet was charged with 1-(6-chloro-3-pyridyl)-2,2,2-trifluoro-ethanone (6.3 g, 30.0 mmol), tert-butyl 6-acetylspiro[1H-isobenzofuran-3,3′-azetidine]-1-carboxylate (4.5 g, 15.0 mmol) and DMF (60 mL). To this mixture was added Cs₂CO₃ (975 mg, 3.0 mmol, 20 mol %) and the reaction was stirred at RT for 10 min. The reaction mixture was poured into water (300 mL), extracted with EtOAc (3×100 mL), washed with LiCl solution (100 mL), dried (Na₂SO₄) and the solvent was evaporated under reduced pressure. The material was placed on a vacuum pump for three days to remove excess 1-(6-chloro-3-pyridyl)-2,2,2-trifluoro-ethanone. The material was then purified by chromatography using a 220 g silica cartridge eluting with heptane-EtOAc, gradient 0 to 20% EtOAc. This chromatographic procedure was repeated a second time affording tert-butyl 6-[(Z)-3-(6-chloro-3-pyridyl)-4,4,4-trifluoro-but-2-enoyl]spiro[1H-isobenzofuran-3,3′- azetidine]-1′-carboxylate as a white solid, 6.9 g, 13.0 mmol, 87% yield. ES LC-MS m/z=439 (M−tBu+H). 1H NMR (CHLOROFORM-d) δ: 8.26 (d, J=2.4 Hz, 1H), 7.88 (d, J=8.0 Hz, 1H), 7.71 (s, 1H), 7.64 (dd, J=8.3, 2.5 Hz, 1H), 7.58 (d, J=8.0 Hz, 1H), 7.51 (d, J=1.4 Hz, 1H), 7.34 (d, J=8.3 Hz, 1H), 5.14 (s, 2H), 4.34 (d, J=9.9 Hz, 2H), 4.13 (d, J=9.9 Hz, 2H), 1.49 (s, 9H).
tert-butyl 6-[3-(6-chloro-3-pyridyl)-4,4,4-trifluoro-3-sulfanyl-butanoyl]spiro[1H-isobenzofuran-3,3′-azetidine]-1′-carboxylate

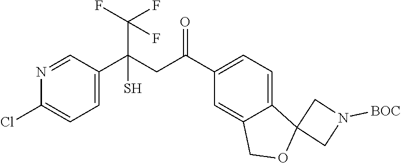 [SMILES: CC(C)(C)OC(=O)N1CC2(C1)OCc1cc(C(=O)CC(S)(c3ccc(Cl)nc3)C(F)(F)F)ccc12]

A round bottom flask equipped with a stir bar and nitrogen inlet was charged with tert-butyl 6-[(Z)-3-(6-chloro-3-pyridyl)-4,4,4-trifluoro-but-2-enoyl]spiro[1H-isobenzofuran-3,3′-azetidine]-1′-carboxylate (6.9 g, 14.0 mmol) 1,4-dioxane (140 mL). Once the material had dissolved, sodium hydrosulphide was dissolved in water (28 mL) and added to the reaction mixture over 5 min. After stirring for 30 min at RT, the mixture was diluted with water (100 mL) and EtOAc (100 mL) and dilute HCl was added until the pH was about 1. The phases were separated and the aqueous was extracted with EtOAc (2×100 mL), the combined organic phases were washed with brine (100 mL), dried (Na₂SO₄) and the solvent was evaporated under reduced pressure. This gave tert-butyl 6-[3-(6-chloro-3-pyridyl)-4,4,4-trifluoro-3-sulfanyl-butanoyl]spiro[1H-isobenzofuran-3,3′-azetidine]-1′-carboxylate as an off- white foam. The material was used as is in the next step assuming 100% yield. ES LC-MS m/z=473 (M−tBu+H).
tert-butyl 6-[5-(6-chloro-3-pyridyl)-5-(trifluoromethyl)-4H-isothiazol-3-yl]spiro[1H-isobenzofuran-3,3′-azetidine]-1′-carboxylate

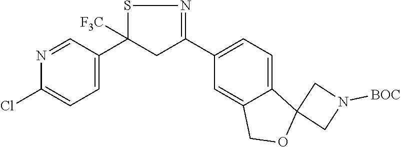 [SMILES: CC(C)(C)OC(=O)N1CC2(C1)OCc1cc(C3=NSC(c4ccc(Cl)nc4)(C(F)(F)F)C3)ccc12]

A round bottom flask equipped with a stir bar, dropping funnel and nitrogen inlet was charged with tert-butyl 6-[3-(6-chloro-3-pyridyl)-4,4,4-trifluoro-3-sulfanyl-butanoyl]spiro[1H-isobenzofuran-3,3′-azetidine]-1′-carboxylate (7.4 g, 14.0 mmol) and DMF (140 mL). The mixture was cooled to −78° C. and a solution of hydroxylamine-O-sulphonic acid (2.4 g, 21.0 mmol) and Hunig's base (12 mL, 70.0 mmol) was added. The reaction was allowed to warm to RT and maintained for 30 min at this temperature. The reaction was poured into saturated LiCl solution (500 mL), extracted with EtOAc (3×150 mL), the combined organic phases were dried (Na₂SO₄) and the solvent was evaporated under reduced pressure. The material was purified by chromatography using a 220 g silica cartridge eluting with heptane-EtOAc, gradient 0 to 20% EtOAc. This purification procedure was repeated obtain tert-butyl 6-[5-(6-chloro-3-pyridyl)-5-(trifluoromethyl)-4H-isothiazol-3-yl]spiro[1H-isobenzofuran-3,3′-azetidine]-1′-carboxylate as a white solid. Amount obtained: 4.4 g, 8.4 mmol, 60% yield. ES LC-MS m/z=470 (M−tBu+H). 1H NMR (CHLOROFORM-d) δ: 8.47 (d, J=2.5 Hz, 1H), 7.75 (d, J=8.0 Hz, 1H), 7.69 (dd, J=8.4, 2.7 Hz, 1H), 7.66 (s, 1H), 7.54 (d, J=8.0 Hz, 1H), 7.40 (d, J=8.4 Hz, 1H), 5.14 (s, 2H), 4.25-4.37 (m, 3H), 4.14 (d, J=9.7 Hz, 2H), 3.91 (d, J=17.5 Hz, 1H), 1.49 (s, 9H).
6-[5-(6-chloro-3-pyridyl)-5-(trifluoromethyl)-4H-isothiazol-3-yl]spiro[1H-isobenzofuran-3,3′-azetidine]

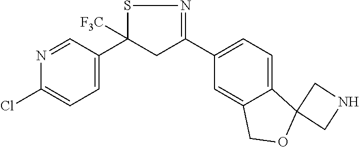 [SMILES: FC(F)(F)C1(c2ccc(Cl)nc2)CC(c2ccc3c(c2)COC32CNC2)=NS1]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (1052 mg, 2.0 mmol) and EtOAc (10 mL). To this mixture was added pTSA and the reaction mixture was stirred for 5 h. The reaction mixture was poured into saturated NaHCO₃ solution (30 mL) and stirred for 30 min. The phases were separated and the aqueous was extracted with EtOAc (3×30 mL), the combined organic phases were dried (Na₂SO₄) and the solvent was evaporated under reduced pressure. The material was used without further purification in the next step. ES LC-MS m/z=426 (M+H+).
Compound 1: 1-[6-[5-(6-chloro-3-pyridyl)-5-(trifluoromethyl)-4H-isothiazol-3-yl]spiro[1 H-isobenzofuran-3,3′-azetidine]-1′-yl]-3,3,3- trifluoro-propan-1-one

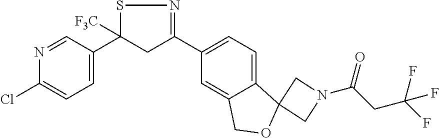 [SMILES: O=C(CC(F)(F)F)N1CC2(C1)OCc1cc(C3=NSC(c4ccc(Cl)nc4)(C(F)(F)F)C3)ccc12]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (213 mg, 0.50 mmol), 3,3,3-trifluoropropanoic acid (64 mg, 0.50 mmol), Hunig's base (0.26 mL, 1.50 mmol) and DMF (10 mL). To this mixture was added 1-[bis(dimethylamino)methylene]-1H-1,2,3-triazolo[4,5-b]pyridinium 3-oxid hexafluorophosphate (HATU) (190 mg, 0.50 mmol). The reaction was stirred at RT for 18 h. The mixture was poured into water (100 mL), the resulting precipitate was filtered and dried under vacuum. The material was purified by chromatography using a 24 g silica cartridge eluting with heptane-EtOAc, gradient 0 to 50% EtOAc. This gave 1-[6-[5-(6-chloro-3-pyridyl)-5-(trifluoromethyl)-4H-isothiazol-3-yl]spiro[1H-isobenzofuran-3,3′-azetidine]-1′-yl]-3,3,3-trifluoro-propan-1-one as a white solid. Amount obtained: 162 mg, 0.30 mmol, 60% yield. ES LC-MS m/z=536 (M+H). 1H NMR (CHLOROFORM-d) δ: 8.47 (d, J=2.6 Hz, 1H), 7.78 (d, J=7.5 Hz, 1H), 7.65-7.73 (m, 2H), 7.48 (d, J=8.0 Hz, 1H), 7.41 (d, J=8.4 Hz, 1H), 5.18 (s, 2H), 4.60 (d, J=9.1 Hz, 1H), 4.39-4.49 (m, 2H), 4.24-4.38 (m, 2H), 3.91 (d, J=17.5 Hz, 1H), 3.08 (qd, J=10.3, 2.3 Hz, 2H).
Compound 2: 1-[6-[5-(6-chloro-3-pyridyl)-5-(trifluoromethyl)-4H-isothiazol-3-yl]spiro[1H-isobenzofuran-3,3′-azetidine]-1′-yl]-2- methylsulfonyl-ethanone

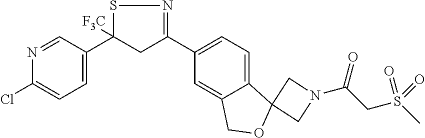 [SMILES: CS(=O)(=O)CC(=O)N1CC2(C1)OCc1cc(C3=NSC(c4ccc(Cl)nc4)(C(F)(F)F)C3)ccc12]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (213 mg, 0.50 mmol), 2-methylsulfonylacetic acid (69 mg, 0.50 mmol), Hunig's base (0.26 mL, 1.50 mmol) and DMF (10 mL). To this mixture was added HATU (190 mg, 0.50 mmol). The reaction was stirred at RT for 18 h. The mixture was poured into water (100 mL), the resulting precipitate was filtered and dried under vacuum. The material was purified by chromatography using a 24 g silica cartridge eluting with heptane-EtOAc, gradient 0 to 100% EtOAc. This gave 1-[6-[5-(6-chloro-3-pyridyl)-5-(trifluoromethyl)-4H-isothiazol-3-yl]spiro[1H-isobenzofuran-3,3′-azetidine]-1′-yl]-2-methylsulfonyl-ethanone as a white solid. Amount obtained: 189 mg, 0.35 mmol, 69% yield. ES LC-MS m/z=546 (M+H). 1H NMR (CHLOROFORM-d) δ: 8.46 (d, J=2.6 Hz, 1H), 7.76 (d, J=8.1 Hz, 1H), 7.66-7.72 (m, 2H), 7.59 (d, J=8.0 Hz, 1H), 7.41 (d, J=8.4 Hz, 1H), 5.18 (s, 2H), 4.64-4.72 (m, 2H), 4.46 (d, J=11.2 Hz, 1H), 4.37 (d, J=11.1 Hz, 1H), 4.28 (d, J=17.5 Hz, 1H), 3.83-3.95 (m, 3H), 3.21 (s, 3H).
Compound 3: 1-[6-[5-(6-chloro-3-pyridyl)-5-(trifluoromethyl)-4H-isothiazol-3-yl]spiro[1H-isobenzofuran-3,3′-azetidine]-1′-yl]-2-methyl- propan-1-one

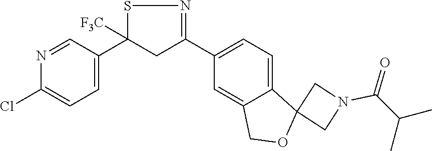 [SMILES: CC(C)C(=O)N1CC2(C1)OCc1cc(C3=NSC(c4ccc(Cl)nc4)(C(F)(F)F)C3)ccc12]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (213 mg, 0.50 mmol), 2-methylpropanoic acid (44 mg, 0.50 mmol), Hunig's base (0.26 mL, 1.50 mmol) and DMF (10 mL). To this mixture was added HATU (190 mg, 0.50 mmol). The reaction was stirred at RT for 18 h. The mixture was poured into water (100 mL), the resulting precipitate was filtered and dried under vacuum. The material was purified by chromatography using a 24 g silica cartridge eluting with heptane-EtOAc, gradient 0 to 100% EtOAc. This gave 1-[6-[5-(6-chloro-3-pyridyl)-5-(trifluoromethyl)-4H-isothiazol-3-yl]spiro[1H-isobenzofuran-3,3′-azetidine]-1′-yl]-2-methyl-propan-1-one as a white solid. Amount obtained : 235 mg, 0.48 mmol, 95% yield. ES LC-MS m/z=496 (M+H). 1H NMR (CHLOROFORM-d) δ: 8.47 (d, J=2.6 Hz, 1H), 7.76 (d, J=8.1 Hz, 1H), 7.65-7.72 (m, 2H), 7.48 (d, J=8.0 Hz, 1H), 7.40 (d, J=8.4 Hz, 1H), 5.17 (s, 2H), 4.54 (d, J=9.0 Hz, 1H), 4.24-4.41 (m, 4H), 3.91 (d, J=17.5 Hz, 1H), 2.46-2.59 (m, 1H), 1.17 (t, J=6.2 Hz, 6H).
Compound 4: 6-[5-(6-chloro-3-pyridyl)-5-(trifluoromethyl)-4H-isothiazol-3-yl]-N-ethyl-spiro[1H-isobenzofuran-3,3′-azetidine]-1′-carboxamide

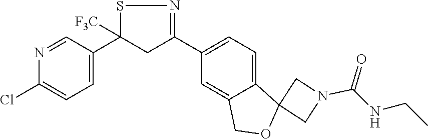 [SMILES: CCNC(=O)N1CC2(C1)OCc1cc(C3=NSC(c4ccc(Cl)nc4)(C(F)(F)F)C3)ccc12]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (213 mg, 0.50 mmol), and CH₂Cl₂ (10 mL). To this solution at RT was added triethylamine (0.21 mL, 1.50 mmol) and ethylisocyanate (36 mg, 0.50 mmol). After stirring for 18 h, the solvent was evaporated under reduced pressure and the material was purified by chromatography using a 24 g silica cartridge eluting with heptane-EtOAc, gradient 0 to 100% EtOAc. This gave 6-[5-(6-chloro-3-pyridyl)-5-(trifluoromethyl)-4H-isothiazol-3-yl]-N-ethyl-spiro[1H-isobenzofuran-3,3′-azetidine]-1′- carboxamide as a white solid. Amount obtained : 223 mg, 0.45 mmol, 90% yield. ES LC-MS m/z=497 (M+H). 1H NMR (CHLOROFORM-d) δ: 8.47 (d, J=2.6 Hz, 1H), 7.76 (d, J=8.0 Hz, 1H), 7.65-7.72 (m, 2H), 7.55 (d, J=8.0 Hz, 1H), 7.40 (d, J=8.4 Hz, 1H), 5.16 (s, 2H), 4.25-4.36 (m, 3H), 4.10-4.19 (m, 3H), 3.91 (d, J=17.5 Hz, 1H), 3.26-3.36 (m, 2H), 1.18 (t, J=7.2 Hz, 3H).
Compound 5: 6-[5-(6-chloro-3-pyridyl)-5-(trifluoromethyl)-4H-isothiazol-3-yl]-1′-ethylsulfonyl-spiro[1H-isobenzofuran-3,3′-azetidine]

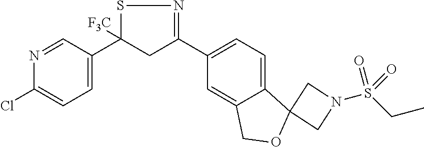 [SMILES: CCS(=O)(=O)N1CC2(C1)OCc1cc(C3=NSC(c4ccc(Cl)nc4)(C(F)(F)F)C3)ccc12]

A scintillation vial equipped with a stir bar and nitrogen inlet was charged with tert-butyl 6-[5-(6-chloro-3-pyridyl)-5-(trifluoromethyl)-4H-isothiazol-3-yl]spiro[1H-isobenzofuran-3,3′-azetidine]-1′-carboxylate (213 mg, 0.50 mmol), and CH₂Cl₂ (10 mL). To this solution at RT was added triethylamine (0.21 mL, 1.50 mmol) and ethylsulphonyl chloride (64 mg, 0.50 mmol) was added. After stirring for 18 h, the reaction mixture was poured into saturated NaHCO3 solution (50 mL). The mixture was extracted with DCM (3×50 mL), the combined organic phases were dried (Na₂SO₄) and the solvent was evaporated under reduced pressure. The material was purified by chromatography using a 24 g silica cartridge eluting with heptane-EtOAc, gradient 0 to 100% EtOAc. This gave 6-[5-(6-chloro-3-pyridyl)-5-(trifluoromethyl)-4H-isothiazol-3-yl]-1′-ethylsulfonyl-spiro[1H-isobenzofuran-3,3′-azetidine] as an off-white solid. Amount obtained : 230 mg, 0.44 mmol, 88% yield. 1H NMR (CHLOROFORM-d) δ: 8.47 (d, J=2.6 Hz, 1H), 7.75-7.79 (m, 1H), 7.65-7.72 (m, 3H), 7.41 (d, J=8.4 Hz, 1H), 5.16 (s, 2H), 4.38 (d, J=9.3 Hz, 2H), 4.29 (d, J=17.5 Hz, 1H), 4.18 (d, J=9.4 Hz, 2H), 3.91 (d, J=17.6 Hz, 1H), 3.09 (q, J=7.4 Hz, 2H), 1.45 (t, J=7.4 Hz, 3H).

### Example 2

1-(6-Chloro-2-pyridyl)-2,2,2-trifluoroethanol

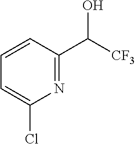 [SMILES: OC(c1cccc(Cl)n1)C(F)(F)F]

A solution of 6-chloropyridine-2-carbaldehyde (1) (4.54 g, 32.1 mmol) in CH₂Cl₂ (50 mL) was chilled to ˜0° C. in an ice/water bath and charged with the dropwise addition of TMSCF₃ (5.47 g, 38.5 mmol), followed by the dropwise addition of tetrabutylammonium fluoride (TBAF) solution (1.0 M in THF, 481 μL, 0.481 mmol). The reaction was allowed to warm to rt and stir for 2 h, and was then charged with the slow addition of additional TBAF solution (1.0 M in THF, 8.00 mL, 8.00 mmol). After stirring at rt for 1 h, the reaction was diluted with EtOAc (500 mL), washed with 0.1 N HCl (1×200 mL), then saturated NaCl solution (1×100 mL), then dried over Na₂SO₄, filtered, and concentrated under reduced pressure. Yield: 6.45 g (95%) as a light tan solid. 1H NMR (400 MHz, CDCl3) δ ppm 4.77 (d, J=7.6 Hz, 1 H), 5.04 (quin, J=6.8 Hz, 1H), 7.38 (d, J=7.6 Hz, 1H), 7.42 (d, J=7.9 Hz, 1H), 7.77 (t, J=7.8 Hz, 1H). MS (M+H) 212.0.
1-(6-Chloro-2-pyridyl)-2,2,2-trifluoroethanone

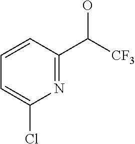 [SMILES: OC(c1cccc(Cl)n1)C(F)(F)F]

A solution of 1-(6-chloro-2-pyridyl)-2,2,2-trifluoroethanol (7.15 g, 33.8 mmol) in CH₂Cl₂ (55 mL) was treated with Dess-Martin periodinane (15.8 g, 37.3 mmol), and allowed to stir at rt for 18 h. The reaction was then diluted with CH₂Cl₂ (250 mL), washed with saturated NaHCO₃ solution (2×100 mL), then saturated NaCl solution (1×100 mL), then dried over Na₂SO₄, filtered, and concentrated under reduced pressure. Crude product was then chromatographed on a 120 g Isco RediSep silica cartridge, eluting with a gradient from 100% heptane to 25:75 EtOAc:heptane, yielding 4.75 g of hydrated product. This material was dissolved in toluene (100 mL) and refluxed with a Dean Stark trap for 15 h to remove water. The resulting toluene solution was concentrated under reduced pressure. Yield: 4.14 g (58%) as a light yellow liquid. 1H NMR (400 MHz, CDCl3) δ ppm 7.66 (dd, J=8.1, 0.8 Hz, 1H), 7.92 (t, J=7.8 Hz, 1H), 8.10 (dd, J=7.6, 0.8 Hz, 1H). MS (M+H2O+H) 228.0.
tert-Butyl-6-[(Z)-3-(6-chloro-2-pyridyl)-4,4,4-trifluorobut-2-enoyl]spiro[1H-isobenzofuran-3,3′-azetidine]-1′-carboxylate

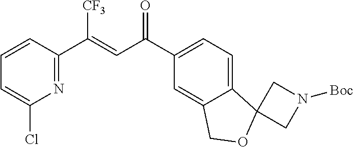 [SMILES: CC(C)(C)OC(=O)N1CC2(C1)OCc1cc(C(=O)/C=C(/c3cccc(Cl)n3)C(F)(F)F)ccc12]

A solution of 1-(6-chloro-2-pyridyl)-2,2,2-trifluoroethanone (4.13 g, 19.7 mmol) in DMF (33 mL) was treated with tert-Butyl 6-acetylspiro[1H-isobenzofuran-3,3′-azetidine]-1′-carboxylate (2.99 g, 9.86 mmol) and Cs2CO3 (0.642 g, 1.97 mmol), and stirred at rt for 20 min. The reaction was then diluted with EtOAc (350 mL) and washed with saturated LiCl solution (2×100 mL), then saturated NaCl solution (1×100 mL), then dried over Na₂SO₄, filtered, and concentrated under reduced pressure, yielding crude product that was predominantly tertiary alcohol intermediate. This material was dissolved in CH₂Cl₂ (50 mL), treated with SOCl₂ (1.70 g, 14.3 mmol) and Et₃N (2.89 g, 28.6 mmol), and stirred at rt for 5 min. The reaction was then chromatographed on a 120 g Isco RediSep silica cartridge, eluting with a gradient from 100% heptane to 50:50 EtOAc: heptane. Yield: 3.41 g (70%) as a yellow oil. 1H NMR (400 MHz, CDCl3) δ ppm 1.49 (s, 9H), 4.13 (d, J=9.3 Hz, 2H), 4.33 (d, J=9.5 Hz, 2H), 5.14 (s, 2H), 7.19 (d, J=8.0 Hz, 1H), 7.23-7.29 (m, 1H), 7.43 (d, J=7.8 Hz, 1H), 7.53 (d, J=8.0 Hz, 1H), 7.66 (t, J=7.9 Hz, 1H), 7.77 (s, 1H), 7.91 (d, J =7.9 Hz, 1H). MS (M−t-Bu+2H) 439.0.
tert-Butyl-6-[3-(6-chloro-2-pyridyl)-4,4,4-trifluoro-3-sulfanylbutanoyl]spiro[1H-isobenzofuran-3,3′-azetidine]-1′-carboxylate

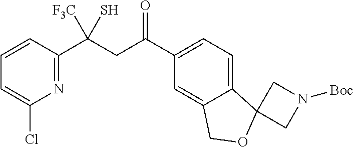 [SMILES: CC(C)(C)OC(=O)N1CC2(C1)OCc1cc(C(=O)CC(S)(c3cccc(Cl)n3)C(F)(F)F)ccc12]

A solution of tert-butyl-6-[(Z)-3-(6-chloro-2-pyridyl)-4,4,4-trifluorobut-2-enoyl]spiro[1H-isobenzofuran-3,3′-azetidine]-1′-carboxylate (2.91 g, 5.88 mmol) in 1,4-dioxane (60 mL) was charged with a solution of NaSH-xH₂O (0.989 g, ˜17.6 mmol) in water (10 mL), and allowed to stir at rt for 90 min. The reaction was then diluted with EtOAc (250 mL) and washed with 0.1 N HCl (3×50 mL), saturated NaCl solution (1×50 mL), then dried over Na₂SO₄, filtered, and concentrated under reduced pressure. Yield: 2.79 g (90%) as a reddish solid. 1H NMR (400 MHz, CDCl3) δ ppm 1.45- 1.58 (m, 9H), 3.63 (d, J=17.5 Hz, 1H), 3.75 (s, 1H), 4.16 (d, J=9.6 Hz, 2H), 4.35 (d, J=9.8 Hz, 2H), 4.81 (d, J=17.5 Hz, 1H), 5.17 (s, 2H), 7.25 (d, J=7.9 Hz, 1H), 7.59 (d, J=8.0 Hz, 1H), 7.74 (t, J=7.9 Hz, 1H), 7.81 (s, 1H), 8.00 (d, J=8.8 Hz, 1H), 8.03 (d, J=7.9 Hz, 1H). MS (M+H) 529.0.
tert-Butyl-6-[5-(6-chloro-2-pyridyl)-5-(trifluoromethyl)-4H-isothiazol-3-yl]spiro[1H-isobenzofuran-3,3′-azetidine]-1′-carboxylate

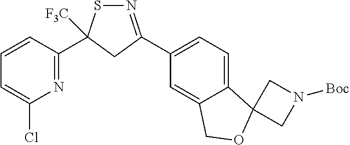 [SMILES: CC(C)(C)OC(=O)N1CC2(C1)OCc1cc(C3=NSC(c4cccc(Cl)n4)(C(F)(F)F)C3)ccc12]

A solution of tert-butyl-6-[3-(6-chloro-2-pyridyl)-4,4,4-trifluoro-3-sulfanylbutanoyl]spiro[1H-isobenzofuran-3,3′-azetidine]-1′-carboxylate (2.29 g, 4.33 mmol) in DMF (35 mL) was briefly chilled in a −78□C dry ice/i-PrOH bath, and charged with a solution of hydroxylamine-O-sulfonic acid (HOSA) (0.734 g, 6.49 mmol) and N,N-diisopropylethylamine (2.80 g, 21.6 mmol) in DMF (10 mL). The reaction was allowed to warm to rt and stir for 45 min, and was then diluted with EtOAc (300 mL), washed with saturated LiCl solution (3×50 mL), then saturated NaCl solution (1×50 mL), dried over Na₂SO₄, filtered, and concentrated under reduced pressure. Crude product was then chromatographed on a 120 g Isco RediSep silica cartridge, eluting with a gradient from 100% heptane to 25:75 EtOAc:heptane. Impure fractions were then chromatographed on a 50 g Isco Gold C18 RediSep reversed phase cartridge, eluting with a gradient from 100% water to 100% MeOH. Yield: 662 mg (29%) as a yellow solid. 1H NMR (400 MHz, CDCl3) δ ppm 1.50 (s, 9H), 4.04 (d, J=18.2 Hz, 1H), 4.15 (d, J=9.5 Hz, 2H), 4.34 (d, J=9.5 Hz, 2H), 4.75 (d, J=18.1 Hz, 1H), 5.15 (s, 2H), 7.36 (d, J=7.9 Hz, 1H), 7.39 (d, J=7.7 Hz, 1H), 7.53 (d, J=8.0 Hz, 1H), 7.69 (s, 1H), 7.73 (t, J =7.8 Hz, 1H), 7.83 (d, J =8.0 Hz, 1H). MS (M−t-Bu+2H) 470.0.
6-[5-(6-Chloro-2-pyridyl)-5-(trifluoromethyl)-4H-isothiazol-3-yl]spiro[1H-isobenzofuran-3,3′-azetidine]

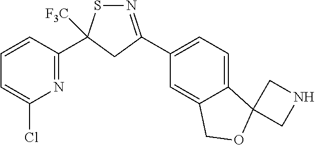 [SMILES: FC(F)(F)C1(c2cccc(Cl)n2)CC(c2ccc3c(c2)COC32CNC2)=NS1]

A solution of tert-butyl-6-[5-(6-chloro-2-pyridyl)-5-(trifluoromethyl)-4H-isothiazol-3-yl]spiro[1H-isobenzofuran-3,3′-azetidine]-1′-carboxylate (100 mg, 0.190 mmol) in EtOAc (2 mL) was charged with p-TsOH-H2O (217 mg, 1.14 mmol), and the reaction stirred at rt for 3.5 h. The reaction was then treated with saturated NaHCO₃ solution (10 mL) and extracted into EtOAc (2×50 mL). Organic fractions were pooled, dried over Na₂SO₄, filtered, and concentrated under reduced pressure. Yield: 81 mg (100%) as a yellow oil. 1H NMR (400 MHz, CDCl3) δ ppm 3.98 (d, J=10.0 Hz, 2H), 4.03 (d, J =18.1 Hz, 1H), 4.31 (d, J=10.0 Hz, 2H), 4.73 (d, J=18.2 Hz, 1H), 5.12 (s, 2H), 7.36 (d, J=7.9 Hz, 1H), 7.39 (d, J=7.8 Hz, 1H), 7.68 (s, 1H), 7.73 (t, J=7.8 Hz, 1H), 7.76-7.81 (m, 1H), 7.83-7.88 (m, 1H). MS (M+H) 426.0.
General procedure for coupling carboxylic acids to 6-[5-(6-chloro-2-pyridyl)-5-(trifluoromethyl)-4H-isothiazol-3-yl]spiro[1 H-isobenzofuran-3,3′-azetidine]. A solution of 6-[5-(6-chloro-2-pyridyl)-5-(trifluoromethyl)-4H-isothiazol-3-yl]spiro[1H-isobenzofuran-3,3′-azetidine] in DMF was charged with appropriate carboxylic acid, HATU, and N,N-diisopropylethylamine, and stirred at rt for 15 h. The reaction was then diluted with EtOAc (100 mL), washed with saturated LiCl solution (2×50 mL), then saturated NaCl solution (1×50 mL), dried over Na₂SO₄, filtered, and concentrated under reduced pressure. Crude product was then chromatographed on a 12 g Isco RediSep silica cartridge, eluting with a gradient from 100% heptane to 100% EtOAc. Product was then chromatographed again on a 15 g Isco Gold C18 RediSep reversed phase cartridge, eluting with a gradient from 100% water to 100% MeOH. Final product was then lyophilized from ˜2 mL ˜1:1 CH3CN:water.
Compound 6: 1-[6-[5-(6-Chloro-2-pyridyl)-5-(trifluoromethyl)-4H-isothiazol-3-yl]spiro[1H-isobenzofuran-3,3′-azetidine]-1′-yl]-3,3,3- trifluoropropan-1-one.

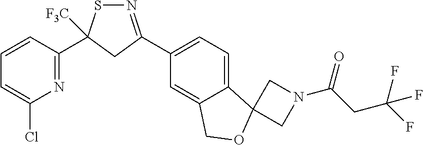 [SMILES: O=C(CC(F)(F)F)N1CC2(C1)OCc1cc(C3=NSC(c4cccc(Cl)n4)(C(F)(F)F)C3)ccc12]

1-[6-[5-(6-Chloro-2-pyridyl)-5-(trifluoromethyl)-4H-isothiazol-3-yl]spiro[1H-isobenzofuran-3,3′-azetidine]-1′-yl]-3,3,3- trifluoropropan-1-one was prepared from 6-[5-(6-chloro-2-pyridyl)-5-(trifluoromethyl)-4H-isothiazol-3-yl]spiro[1H-isobenzofuran- 3,3′-azetidine] (81 mg, 0.19 mmol), 3,3,3-trifluoropropanoic acid (24 mg, 0.19 mmol), HATU (72 mg, 0.19 mmol), and N,N-diisopropylethylamine (74 mg, 0.57 mmol) in DMF (5 mL). Yield: 32 mg (31%) as a white solid. 1H NMR (400 MHz, CDCl3) δ ppm 3.08 (qd, J=10.3, 2.2 Hz, 2H), 4.03 (d, J=18.2 Hz, 1H), 4.30-4.38 (m, 1H), 4.39-4.50 (m, 2H), 4.60 (d, J=9.0 Hz, 1H), 4.75 (d, J=18.0 Hz, 1H), 5.18 (s, 2H), 7.33-7.43 (m, 2H), 7.48 (d, J=8.0 Hz, 1H), 7.67-7.79 (m, 2H), 7.85 (d, J=7.8 Hz, 1H). MS (M+H) 536.0.
Compound 7: 1-[6-[5-(6-Chloro-2-pyridyl)-5-(trifluoromethyl)-4H-isothiazol-3-yl]spiro[1H-isobenzofuran-3,3′-azetidine]-1′-yl]-2- methylsulfonylethanone.

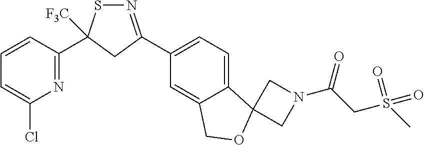 [SMILES: CS(=O)(=O)CC(=O)N1CC2(C1)OCc1cc(C3=NSC(c4cccc(Cl)n4)(C(F)(F)F)C3)ccc12]

1-[6-[5-(6-Chloro-2-pyridyl)-5-(trifluoromethyl)-4H-isothiazol-3-yl]spiro[1H-isobenzofuran-3,3′-azetidine]-1′-yl]-2- methylsulfonylethanone was prepared from 6-[5-(6-chloro-2-pyridyl)-5-(trifluoromethyl)-4H-isothiazol-3-yl]spiro[1H-isobenzofuran-3,3′-azetidine] (81 mg, 0.19 mmol), 2-methylsulfonylacetic acid (26 mg, 0.19 mmol), HATU (72 mg, 0.19 mmol), and N,N-diisopropylethylamine (74 mg, 0.57 mmol) in DMF (5 mL). Yield: 32 mg (31%) as a white solid. 1H NMR (400 MHz, CDCl3) δ ppm 3.21 (s, 3H), 3.88 (s, 2H), 4.03 (d, J=18.2 Hz, 1H), 4.33-4.52 (m, 2H), 4.68 (d, J=3.5 Hz, 2H), 4.74 (d, J=18.2 Hz, 1H), 5.18 (s, 2H), 7.38 (dd, J=10.9, 8.0 Hz, 2H), 7.58 (d, J=8.1 Hz, 1H), 7.67-7.77 (m, 2H), 7.84 (d, J=8.1 Hz, 1H). MS (M+H) 545.8.
Compound 8: 1-[6-[5-(6-Chloro-2-pyridyl)-5-(trifluoromethyl)-4H-isothiazol-3-yl]spiro[1 H-isobenzofuran-3,3′-azetidine]-1′-yl]-2- methylpropan-1-one.

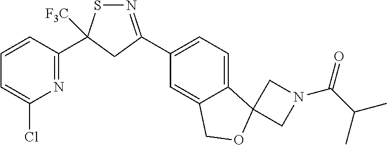 [SMILES: CC(C)C(=O)N1CC2(C1)OCc1cc(C3=NSC(c4cccc(Cl)n4)(C(F)(F)F)C3)ccc12]

1-[6-[5-(6-Chloro-2-pyridyl)-5-(trifluoromethyl)-4H-isothiazol-3-yl]spiro[1H-isobenzofuran-3,3′-azetidine]-1′-yl]-2-methylpropan-1-one was prepared from 6-[5-(6-chloro-2-pyridyl)-5-(trifluoromethyl)-4H-isothiazol-3-yl]spiro[1H-isobenzofuran-3,3′-azetidine] (81 mg, 0.19 mmol), 2-methylpropanoic acid (17 mg, 0.19 mmol), HATU (72 mg, 0.19 mmol), and N,N-diisopropylethylamine (74 mg, 0.57 mmol) in DMF (5 mL). Yield: 60 mg (64%) as a white solid. 1H NMR (400 MHz, CDCl3) δ ppm 1.18 (t, J=6.8 Hz, 6H), 2.53 (dt, J=13.6, 6.8 Hz, 1H), 4.03 (d, J=18.2 Hz, 1H), 4.22-4.32 (m, 1H), 4.37 (t, J=11.0 Hz, 2H), 4.54 (d, J=9.0 Hz, 1H), 4.75 (d, J=18.0 Hz, 1H), 5.18 (s, 2H), 7.32-7.43 (m, 2H), 7.47 (d, J=8.0 Hz, 1H), 7.67-7.78 (m, 2H), 7.83 (d, J=8.0 Hz, 1H). MS (M+H) 496.0.
General procedure for coupling sulfonyl halide or isonitrile to 6-[5-(6-chloro-2-pyridyl)-5-(trifluoromethyl)-4H-isothiazol-3-yl]spiro[1H-isobenzofuran-3,3′- azetidine] to prepare sulfonamide or urea analogs. A solution of 6-[5-(6-chloro-2-pyridyl)-5-(trifluoromethyl)-4H-isothiazol-3-yl]spiro[1H-isobenzofuran-3,3′-azetidine] in CH₂Cl₂ was charged with appropriate sulfonyl halide or isonitrile and Et₃N, and stirred at rt for 15 h. The reaction was then chromatographed on a 12 g Isco RediSep silica cartridge, eluting with a gradient from 100% heptane to 100% EtOAc. Product was then chromatographed again on a 15 g Isco Gold C18 RediSep reversed phase cartridge, eluting with a gradient from 100% water to 100% MeOH. Final product was then lyophilized from ˜2 mL ˜1:1 CH3CN:water.
Compound 9: 6-[5-(6-Chloro-2-pyridyl)-5-(trifluoromethyl)-4H-isothiazol-3-yl]-N-ethylspiro[1H-isobenzofuran-3,3′-azetidine]-1′-carboxamide

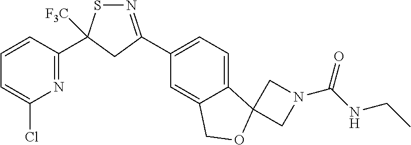 [SMILES: CCNC(=O)N1CC2(C1)OCc1cc(C3=NSC(c4cccc(Cl)n4)(C(F)(F)F)C3)ccc12]

6-[5-(6-Chloro-2-pyridyl)-5-(trifluoromethyl)-4H-isothiazol-3-yl]-N-ethylspiro[1H-isobenzofuran-3,3′-azetidine]-1′-carboxamide was prepared from 6-[5-(6-chloro-2-pyridyl)-5-(trifluoromethyl)-4H-isothiazol-3-yl]spiro[1H-isobenzofuran-3,3′-azetidine] (81 mg, 0.19 mmol), ethyl isocyanate (14 mg, 0.19 mmol) and Et3N (58 mg, 0.57 mmol) in CH₂Cl₂ (5 mL). Yield: 52 mg (55%) as a white solid. 1H NMR (400 MHz, CDCl3) δ ppm 1.18 (t, J=7.2 Hz, 3H), 3.22-3.41 (m, 2H), 4.03 (d, J=18.1 Hz, 1H), 4.10-4.22 (m, 3H), 4.34 (d, J=8.6 Hz, 2H), 4.74 (d, J=18.1 Hz, 1H), 5.16 (s, 2H), 7.33-7.43 (m, 2H), 7.55 (d, J=8.0 Hz, 1H), 7.70 (s, 1H), 7.73 (t, J=7.8 Hz, 1H), 7.83 (d, J=8.0 Hz, 1H). MS (M+H) 497.0.
Compound 10: 6-[5-(6-Chloro-2-pyridyl)-5-(trifluoromethyl)-4H-isothiazol-3-yl]-1′-ethylsulfonylspiro[1H-isobenzofuran-3,3′-azetidine].

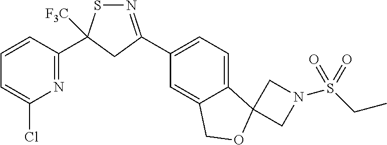 [SMILES: CCS(=O)(=O)N1CC2(C1)OCc1cc(C3=NSC(c4cccc(Cl)n4)(C(F)(F)F)C3)ccc12]

6-[5-(6-Chloro-2-pyridyl)-5-(trifluoromethyl)-4H-isothiazol-3-yl]-1′-ethylsulfonylspiro[1H-isobenzofuran-3,3′-azetidine] was prepared from 6-[5-(6-chloro-2-pyridyl)-5-(trifluoromethyl)-4H-isothiazol-3-yl]spiro[1H-isobenzofuran-3,3′-azetidine] (81 mg, 0.19 mmol), ethanesulfonyl chloride (24 mg, 0.19 mmol) and Et3N (58 mg, 0.57 mmol) in CH₂Cl₂ (5 mL). Yield: 57 mg (58%) as a white solid. 1H NMR (400 MHz, CDCl3) δ ppm 1.46 (t, J=7.4 Hz, 3H), 3.10 (q, J=7.4 Hz, 2H), 4.03 (d, J=18.1 Hz, 1H), 4.18 (d, J=9.3 Hz, 2H), 4.39 (d, J=9.2 Hz, 2H), 4.75 (d, J=18.2 Hz, 1H), 5.16 (s, 2H), 7.32-7.44 (m, 2H), 7.65-7.71 (m, 2H), 7.74 (t, J=7.8 Hz, 1H), 7.85 (d, J=8.2 Hz, 1H). MS (M+H) 517.8.

### Example 3

1-(3,5-dichlorophenyl)-2,2,2-trifluoroethanone

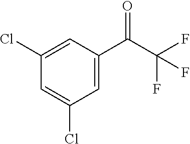 [SMILES: O=C(c1cc(Cl)cc(Cl)c1)C(F)(F)F]

To a solution of 1-bromo-3,5-dichlorobenzene (25.0 g, 110.6 mmol) in 400 ml THF at room temperature, isopropyl magnesium chloride lithium chloride complex (85.0 ml, 1.3M THF, 110.6 mmol) was added dropwise at room temperature over a period of 15 minutes while holding the reaction temperature between 20° C. and 25° C. After the addition was complete, the reaction was allowed to stir 1.5 hours at room temperature. The reaction solution was then cooled to −5° C. to −10° C. with ice/MeOH. Methyltrifluoroacetate (12.23 ml, 121.6 mmol) in 20 ml THF was added dropwise to the reaction solution while maintaining the reaction temp below 0° C. (˜30 min). The reaction was stirred at −5° C. for 0.5 hr then was allowed to warm to room temperature and stir for 1.5 hrs. The reaction was cooled again to −5° C. to −10° C. then 73.7 ml 6M HCl diluted to 150 ml total volume water was added dropwise while keeping the temperature below 0° C. Once the addition was complete, the reaction was stirred 0.5 hr at 0° C. then was allowed to warm to room temperature. Excess water was added and the resulting organic layer that separated was drawn off. The aqueous layer was washed repeatedly with DCM. The combined DCM washes and recovered organic layer were dried over sodium sulfate and concentrated to yield 90 g of an oil.

The crude material was then passed through a silica gel plug (neat heptane to neat DCM) and purified by vacuum distillation (3 torr, product fractions recovered from 112° C. to 125° C.) to provide 20.3 g product (75.7% yield).

1H NMR (400 MHz, CHLOROFORM-d) δ ppm 7.71 (s, 1 H) 7.93 (s, 2 H). IR 1728.1 cm⁻¹
tert-Butyl 6[(Z)-3-(3,5-dichloro-phenyl)-4,4,4,-trifluoro-but-2-enoyl]spiro[1H-isobenzofuran-3,3′-azetidine]-1′-carboxylate

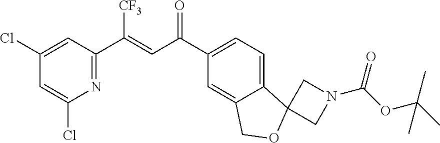 [SMILES: CC(C)(C)OC(=O)N1CC2(C1)OCc1cc(C(=O)/C=C(/c3cc(Cl)cc(Cl)n3)C(F)(F)F)ccc12]

tert-Butyl 6-acetyspiro[-1H-isobenzofuran-3.3′-azetidine]1′-carboxylate (500 mg, 1.64 mmol) and cesium carbonate (53.5 mg, 0.163 mmol) were combined in 5 ml 1:1 trifluoromethylbenzene:toluene. 1-(3,5-dichlorophenyl)-2,2,2-trifluoroethanone (438.3 mg, 1.80 mmol) was added and the mixture was brought to 100° C. and was stirred 1.5 hrs. TLC (3:7 EtOAc: Heptane) indicated incomplete consumption of starting material. More 1-(3,5-dichlorophenyl)-2,2,2-trifluoroethanone (100.0 mg, 0.41 mmol) and cesium carbonate was added and stirring was continued for 1.5 hrs. This was repeated one more time and allowed to stir overnight at 100° C. The reaction was filtered, concentrated, and chromatographed (Isco Companion, 24 g SiO2 cartridge, 100% DCM to 100% EtOAc over 30 min @ 35 ml/min) to afford 761.0 mg, 87.8% yield) yellow foamy solid. 1H NMR (400 MHz, DMSO-d6) δ ppm 1.39-1.47 (m, 9 H) 4.07-4.19 (m, 4 H) 5.09 (s, 2 H) 7.36 (d, J=1.85 Hz, 2 H) 7.68 (dt, J=3.90, 2.05 Hz, 2 H) 7.84-7.89 (m, 2 H) 7.90-7.95 (m, 1 H). LCMS [M-1]=527.8
tert-Butyl 6-[5-(3,5-dichloro-phenyl)-5-(trifluoromethyl)-4H-isothiazol-3-yl]spiro[1H-isobenzofuran-3-3′-azetidine]-1′carboxylate

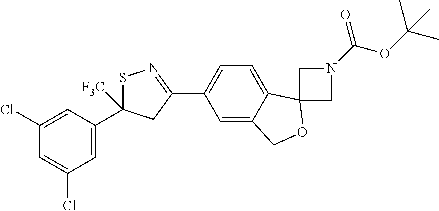 [SMILES: CC(C)(C)OC(=O)N1CC2(C1)OCc1cc(C3=NSC(c4cc(Cl)cc(Cl)c4)(C(F)(F)F)C3)ccc12]

To a solution of tert-butyl 6[(Z)-3-(3,5-dichloro-phenyl)-4,4,4,-trifluoro-but-2-enoyl]spiro[1H-isobenzofuran-3,3′-azetidine]-1′-carboxylate (761.0 mg, 1.44 mmol) in 12 ml dioxane, was added NaSH in 2 ml water and the resulting solution was stirred 0.5 hr at RT. The reaction solution was then poured into a separatory funnel containing EtOAc and washed with 1M HCl (1×10 ml) and brine (2×20 ml), dried over sodium sulfate, and concentrated to give a white foamy solid. This solid was immediately dissolved in 7 ml THF. Solid HOSA was added followed by 19 ml 0.3 M KOH solution that had been cooled to 0° C. The resulting mixture was allowed to stir 10 min at room temperature then 10 ml 1M HCl was added slowly. The aqueous mixture was then allowed to stir 10 min and was poured into a separatory funnel containing EtOAc. The organic layer was separated and washed with 2×20 ml brine, dried over sodium sulfate, chromatographed (Isco Companion, 40 g SiO2 cartridge, neat DCM 5 min, neat DCM to neat EtOAc over 40 min @ 40 ml/min) to afford 609.6 mg (75.6% yield) of a light yellow solid. 1H NMR (400 MHz, CHLOROFORM-d) δ ppm 1.48-1.52 (m, 9 H) 3.98 (d, J=18.35 Hz, 1 H) 4.15 (d, J=9.96 Hz, 2 H) 4.31 (d, J=18.35 Hz, 1 H) 4.36 (d, J=10.05 Hz, 2 H) 5.17 (s, 2 H) 7.35 (t, J=1.78 Hz, 1 H) 7.56 (d, J=0.93 Hz, 2 H) 7.61 (d, J=7.96 Hz, 1 H) 7.78 (s, 1 H) 7.97 (d, J=8.00 Hz, 1 H). LCMS [M-1]=559.8
Compound 11: 6-[5-(3,5-dichlorophenyl)-5-(trifluoromethyl)-4H-isothiazol-3-yl]-N-ethyl-spiro[1H-isobenzofuran-3,3′-azetidine]-1′-carboxamide

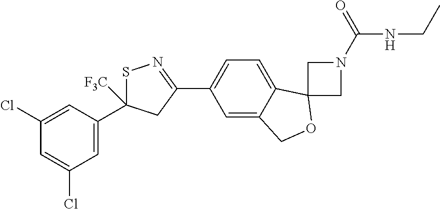 [SMILES: CCNC(=O)N1CC2(C1)OCc1cc(C3=NSC(c4cc(Cl)cc(Cl)c4)(C(F)(F)F)C3)ccc12]

To tert-butyl 6-[5-(3,5-dichloro-phenyl)-5-(trifluoromethyl)-4H-isothiazol-3-yl]spiro[1H-isobenzofuran-3-3′-azetidine]-1′carboxylate (50.0 mg, 0.0893 mmol) was added 5.0 ml 1:1 TFA:DCM and the resulting clear colorless solution was stirred for 15 min at room temperature. The TFA:DCM solution was evaporated and the crude was dried on high vacuum line for 2 hrs. The crude material was dissolved in 3 ml DCM. EtNCO (33.6 ul, 0.283 mmol) was added followed by pyridine (22.8 ul, 0.283 mmol) and the reaction was allowed to stir 20 min. The reaction solution was then coated onto celite and chromatographed (Isco Companion, 13 g RP-18 cartridge, 20:80 water:acetonitrile with 0.01% HCl to neat acetonitrile over 40 min @30 ml/min) to afford 19.0 mg (40.1% yield) of a white solid. 1H NMR (400 MHz, CHLOROFORM-d) δ ppm 1.18 (t, J=7.22 Hz, 3 H) 3.27-3.36 (m, 3 H) 3.98 (d, J=18.35 Hz, 1 H) 4.14 (d, J=8.49 Hz, 2 H) 4.19 (br. s., 1 H) 4.31 (d, J=18.35 Hz, 1 H) 4.35 (d, J=8.44 Hz, 2 H) 5.18 (s, 2 H) 7.34-7.37 (m, 1 H) 7.56 (s, 2 H) 7.63 (d, J=8.00 Hz, 1 H) 7.79 (s, 1 H) 7.97 (d, J=7.96 Hz, 1 H). LCMS [M+H]=533, [M-1]=530.8.
Compound 12: 1-[6-[5-(3,5-dichlorophenyl)-5-(trifluoromethyl)-4H-isothiazol-3-yl]spiro[1 H-isobenzofuran-3,3′-azetidine]-1′-yl]-3,3,3- trifluoro-propan-1-one To a solution of 6-[5-(3,5-dichlorophenyl)-5-(trifluoromethyl)-4H-isothiazol-3-yl]spiro[1H-isobenzofuran-3,3′-azetidine] (˜10 mg, 0.023 mmol) in DMF (2 mL) was added hydroxybenzotriazole (HOBt) (32 mg, 0.25 mmol), diisopropylethylamine (−59 mg, 0.5 mmol), and 3,3,3-trifluoropropanoic acid (27 mg, 0.23 mmol) followed by 1- ethyl-3-(3-dimethylaminopropyl)carbodiimide (EDCl) (43 mg, 0.23 mmol). The reaction mixture was stirred at room temperature for 18 hours. Several drops of water were added, and the mixture was purified by reverse phase HPLC (MeOH/water) to afford the product as the white solid (6 mg, 45%): LCMS m/e 569 (M+H); 1H NMR (400 MHz, METHANOL-d4) δ ppm 3.31-3.28 (m, 2 H) 4.11 (d, J=18.21 Hz, 1 H) 4.25-4.35 (m, 2 H) 4.42 (d, J=18.11 Hz, 1 H) 4.50-4.63 (m, 2 H) 5.15 (s, 2 H) 7.44 (d, J=1.07 Hz, 2 H) 7.54 (t, J=1.78 Hz, 1 H) 7.59 (d, J=8.05 Hz, 1 H) 7.77 (s, 1 H) 7.90 (d, J=8.00 Hz, 1 H)

By following similar coupling procedures to Examples 1-3, compounds 13-15 may be prepared.

### Example 4

tert-Butyl 6-[(E/Z)-3-(3,5-dichlorophenyl)but-2-enoyl]spiro[1H-isobenzofuran-3,3′-azetidine]-1′-carboxylate

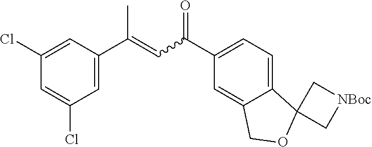 [SMILES: CC(=CC(=O)c1ccc2c(c1)COC21CN(C(=O)OC(C)(C)C)C1)c1cc(Cl)cc(Cl)c1]

TiCl₄ (2.91 mL, 2.91 mmol, 1M in DCM) and tributylamine (0.76 mL, 3.17 mmol) were successively added to a stirred solution of 1-(3,5-dichlorophenyl)ethanone (0.5 g, 2.65 mmol) in DCM (6 mL) at −78° C. After 30 minutes, tert-butyl 6-acetylspiro[1H-isobenzofuran-3,3′-azetidine]-1′-carboxylate (0.84g, 2.78 mmol) in DCM (2 mL) was added to the mixture at −78° C. Excess dry ice were removed and the reaction gradually warmed to −45° C. and stirred between −42° C. and −48° C. for 1 h. Pyridine (1.16 mL, 14.36 mmol) was added at −45° C., the cold bath removed and the reaction continued at RT overnight. It was diluted with water and extracted with DCM (3×50 mL), washed with brine, dried (Na₂SO₄), filtered and evaporated. The material was purified by chromatography, 80 g cartridge, eluting with 0-20% EtOAc/Heptane to give a 1:6 isomeric mixture of cis and trans oils. Combined yield 0.62 g (50.2%). Trans isomer (major): 1H NMR (400 MHz, CDCl3) δ: 7.82 (d, J=1.9 Hz, 2H), 7.52-7.60 (m, 3H), 7.40 (s, 1H), 7.01-7.04 (m, 1H), 5.18 (s, 2H), 4.35 (d, J=9.5 Hz, 2H), 4.17 (d, J=9.5 Hz, 2H), 2.62 (d, J=1.2 Hz, 3H), 1.50 (s, 9H); LCMS-ELSD tR=2.040 min, (M+H) 418.0. Cis isomer (minor): 1H NMR (400 MHz, CDCl3) δ: 7.62 (d, J=1.9 Hz, 2H), 7.42 (t, J=1.9 Hz, 1H), 7.37 (d, J=7.8 Hz, 1H), 7.18 (s, 1H), 7.00 (s, 1H), 6.64-6.68 (m, 1H), 5.05 (s, 2H), 4.29 (d, J=9.5 Hz, 2H), 4.10 (d, J=9.6 Hz, 2H), 2.34 (d, J=1.3 Hz, 3H), 1.48 (s, 9H); LCMS-ELSD tR=1.833 min, (M+H) 418.0. By proceeding in a similar fashion to examples 1-4, compounds 36-40 may be prepared.

### Evaluation

The biological activity of compounds of the present invention was tested using the test methods described below.

R. sanguineus, D. variablis, A. americanum, and I. scapularis Residual Adult Contact Assay

A solution of the test compounds was used to coat the inner wall of glass vials and to treat two filter papers. Once dried, one filter paper was placed in the cap of the vial and the other in the bottom of the vial. Each treated vial was infested with 10 ticks. Contact of the ticks with residues was induced by holding the vials in a controlled environment and assessment was performed at 12, 24 and 48 hours after application in comparison with untreated glass vials and solvent-treated glass vials. Compounds were tested in duplicate and two separate trials (n=4).

Preferred compounds of the invention are generally active at below 500 ppm at 48 hours in the assay.

Particularly preferred compounds are active at below 50 ppm at 48 hours in this assay.

Ctenocephalides felis Residual Contact Assay

A solution of the test compounds, individually and in combination was dispensed onto a substrate placed into a glass vial. The treated surface was allowed to dry before infesting each vial with 10 adult Ctenocephalides felis. The treated vials were held in a controlled environment and assessment was performed at 6, 24 and 48 hours after application in comparison with untreated controls glass vials and solvent-treated glass vials. Compounds were tested in duplicate and two separate trials (n=4).

Preferred compounds of the invention are generally active at below 500 ppm at 48 hours in the assay.

Particularly preferred compounds are active at below 50 ppm at 48 hours in this assay.

In Vitro Evaluation of Ingestion Activity Against Fleas (Ctenocephalides felis)

For flea ingestion tests, an aliquot of each compound stock was added to organic bovine blood contained in an artificial feeding container, with a final DMSO concentration of 0.5%. Ten newly emerged unfed fleas, Ctenocephalides felis, from a laboratory colony, 0-7 days old, were aspirated into a chamber and exposed to the appropriate feeding container and held in a controlled environment at 35° C. Fleas were evaluated for % mortality at 24, 48, and 72 hours post infestation. Fleas showing normal movement and/or jumping ability were considered viable and those showing no movement after tapping the vials were scored as dead. The compound+blood mixture was replaced every 24 hours.

Preferred compounds of the invention are generally active at below 500 ppm at 24 hours in this assay.

Particularly preferred compounds are active at below 50 ppm at 48 hours in this assay.

Dirofilaria immitis Microfilariae Motility Assay

Dirofilaria immitis microfilariae are isolated by filtration from blood of an infected beagle dog allowed to incubate at 37 C/5% CO₂/95% RH in RPMI media. For assay 500 microfilariae are added into 96-well plates followed by addition of compounds diluted in DMSO for single-point or dose response (5-point) analysis. Ivermectin or emodepside are included as a positive control and DMSO-only wells are included as negative controls. Plates containing parasites and compounds are incubated at 37° C./5% CO₂/95% RH for 72 hours and motility is assessed using an LCD camera imaging system. Percent motility inhibition values are generated relative to the average of the DMSO-only wells. For dose response analysis, data points were averaged and curve fitting software is used to generate sigmoidal curves for the determination of EC₅₀ values (i.e. the effective concentration to kill 50% of the organism).

Preferred compounds of the invention are generally active at below 100 μM in this assay.

Particularly preferred compounds are active below 10 μM.

In Vitro Evaluation of Ingestion Activity Against Mosquitoes (Aedes aegypit)

A solution of test compounds is added to bovine blood contained in a feeding chamber to reach the desired final concentration. Blood spiked with DMSO is prepared to serve as control. Adult female mosquitoes are introduced at the bottom of the chamber and allowed to feed on blood mixtures containing DMSO or test compounds for 30 minutes. Fully engorged mosquitoes are sorted into clean chambers after the blood meal and monitored for survival over 3 days. Replicates containing 10 mosquitoes are performed with each test compound concentration and results calculated as % mortality at specific time points.

Preferred compounds of the invention are generally active at below 500 ppm at 20 hours in this assay.

Particularly preferred compounds are active at below 1 ppm at 20 hours in this assay.

In Vitro Evaluation of Contact Activity Against Mosquitoes (Aedes aegypti)

A solution of the test compound is used to coat the inner walls of glass vials and allowed to dry overnight. Five female Aedes aegypti adults are added to each vial. Contact of the mosquitoes is induced by holding the vials in a controlled environment and assessment of mortality/knockdown is performed at 24 hours after application in comparison to solvent-treated glass vials. Compounds are tested in duplicate on a total of 10 mosquitoes/treatment dose.

Preferred compounds of the invention are generally active at below 500 ppm at 48 hours in the assay.

In Vitro Evaluation of Contact Activity Against Flies (Stomoxys calcitrans)

A solution of the test compounds is added to a piece of filter paper embedded in a petri dish and allowed to dry overnight. Ten 2-3 day old adult flies are added to each petri dish and a sucrose-soaked dental wick is added as a food source. Flies are held at room temperature and assessed for mortality/knockdown at desired time points.

Preferred compounds of the invention are generally active at below 500 ppm at 48 hours in the assay.

All publications, patents and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula (I): 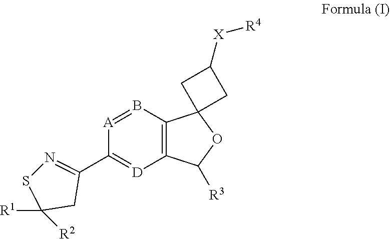
wherein:
R¹ is optionally substituted aryl or optionally substituted heteroaryl;
R² is alkyl or haloalkyl;
each of A, B, and D individually is CR⁵ or N;
each R⁵ individually is hydrogen, alkyl, halogen, haloalkyl, or aryl;
X is L¹-L²-L³;
each of L¹, L², and L³ individually is bond, C(O), (CH₂)ₙ, SO₂, O, or NH;
n is 1 to 6;
R³ is hydrogen, alkyl, or ═O; and
R⁴ is hydrogen, optionally substituted alkyl, optionally substituted haloalkyl, optionally substituted cycloalkyl, optionally substituted heterocyclyl, optionally substituted aryl, or optionally substituted heteroaryl;
or a pesticidally, veterinarily, or pharmaceutically acceptable salt thereof.

2. The compound according to claim 1, wherein:
R¹ is aryl optionally substituted with 1-3 substituents selected from halogen, haloalkyl, or alkyl; or
R¹ is heteroaryl optionally substituted with 1-3 substituents selected from halogen, haloalkyl, or alkyl;

3. The compound according to claim 1, wherein:
R² is CF₃ or CH₃.

4. The compound according to claim 1, wherein:
i) each of A, B, and D is CH;
ii) A is CH, B is N, and D is CH; or
iii) A is CH, B is CH, and D is N;

5. The compound according to claim 1, wherein:
i) L¹ is C(O), L² is (CH₂)ₙ and n is 1, and L³ is bond;
ii) L¹ is C(O), L² is (CH₂)ₙ and n is 1, and L³ is SO₂;
iii) L¹ is C(O), L² is bond, and L³ is bond;
iv) L¹ is C(O), L² is NH, and L³ is (CH₂)ₙ and n is 1;
v) L¹ is SO₂, L² is (CH₂)ₙ and n is 1, and L³ is bond

6. The compound according to claim 1, wherein:
R³ is hydrogen or ═O.

7. The compound according to claim 1, wherein:
R⁴ is C₁₋₆ alkyl or C₁₋₆ haloalkyl,

8. (canceled)

9. A combination comprising:
a compound of claim 1; and
one or more other pesticidally, veterinarily, or pharmaceutically active substances.

10. A composition comprising a compound of claim 1 and an acceptable carrier.

11. A method for controlling parasites at a locus which comprises applying to the locus an effective amount of a composition of claim 10.

12. A method of treating or preventing parasitic infection or infestation in a subject, the method comprising administering to the subject an effective amount of a compound of claim 1.

13. (canceled)

14. The method of claim 12, wherein the parasite is a flea or tick.

15. The method of claim 12, wherein the parasite is Ctenocephalides felis, R. sanguineus, D. variablis or I. scapularis.

16. The method of claim 12, wherein the parasite is a helminth.

17. The method of claim 12, wherein the parasite is Dirofilaria immitis.

18. (canceled)
